(19) United States
(12) Patent Application Publication
(10) Pub. No.: US 2026/0295059 A1
(43) Pub. Date: Oct. 1, 2026

(54) BROAD-SPECTRUM ANTIBIOTIC HYBRIDS FOR THE TREATMENT OF MULTI-DRUG RESISTANT INFECTIONS

(71) Applicants: THE UNIVERSITY OF NORTH CAROLINA AT CHAPEL HILL, Chapel Hill, NC (US); MONASH UNIVERSITY, Clayton, Victoria (AU)

(72) Inventors: Gauri Rao, Chapel Hill, NC (US); Rajnikant Sharma, Chapel Hill, NC (US); Tony Velkov, Geelong West, Victoria (AU); Jian Li, Carnegie, Victoria (AU); Varsha Thombare, Mulgrave, Victoria (AU); Nitin Aniruddha Patil, Morrisville, NC (US)

(21) Appl. No.: 19/477,589

(22) PCT Filed: Apr. 25, 2024
(86) PCT No.: PCT/US2024/026221

§ 371 Date: Oct. 22, 2025

Related U.S. Application Data
(60) Provisional application No. 63/498,184, filed on Apr. 25, 2023.
(60) Provisional application No. 63/513,434, filed on Jul. 13, 2023.

(51) Int. Cl. A61K 47/64; A61K 9/00; A61K 9/06; A61K 38/12; A61K 47/69; A61P 31/04

(52) U.S. Cl. CPC A61K 47/64; A61K 9/0014; A61K 9/0075; A61K 9/06; A61K 38/12; A61K 47/6903; A61K 47/6941; A61P 31/04

(57) ABSTRACT

Provided herein according to some embodiments a hybrid antibiotic molecule that may be resistant to multi-drug resistance. Dry powder and hydrogel formulations of the hybrid antibiotic molecules are disclosed. Also provided herein is a method of treating a bacterial infection in a subject in need thereof, comprising administering to the subject a therapeutically effective amount of the hybrid antibiotic molecule or a pharmaceutical composition comprising the hybrid antibiotic molecule, thereby treating the bacterial infection in the subject.

### STATEMENT OF PRIORITY

This application claims the benefit of U.S. Provisional Application Ser. No. 63/498,184, filed Apr. 25, 2023, and 63/513,434, filed Jul. 13, 2023, the entire contents of which are incorporated by reference herein.

### GOVERNMENT SUPPORT

This invention was made with government support under Grant Number W81XWH2010500, awarded by the Department of Defense and Grant Number AI170889 awarded by the National Institutes of Health. The government has certain rights in this invention.

### STATEMENT REGARDING ELECTRONIC FILING OF A SEQUENCE LISTING

A Sequence Listing in XML format, entitled 5470-948WO_ST26.xml, 68,284 bytes in size, generated on Apr. 23, 2024, and filed herewith, is hereby incorporated by reference in its entirety for its disclosures.

### FIELD OF THE INVENTION

This invention relates to antibiotic compounds and compositions. Teixobactin-based antibiotic hybrids are provided. The invention also relates to methods of using the teixobactin-based antibiotic compounds, including in the treatment of drug resistant infections.

### BACKGROUND

Antibiotics are one of the pillars of modern medicine. However, we are now losing the race between antibiotic drug development and the ability of bacteria to develop resistance. The World Health Organization (WHO) has identified antimicrobial resistance as one of the three greatest threats to human health. The cost of antibiotic resistance, including in chronic wound care, is staggering. Wound care can result in lengthy hospitalisations because of infections caused by pan-drug resistant (PDR) bacterial pathogens that are resistant to every available antibiotic class. There is an urgent unmet medical need for new antibiotics for PDR bacterial pathogens, including in lung infections and wound and burn infections. Infections caused by PDR ‘superbugs,’ particularly Gram-negative Pseudomonas aeruginosa, Acinetobacter baumannii, Klebsiella pneumoniae and Gram-positive Staphylococcus aureus, are of particular concern because of outbreaks of infections caused by PDR strains of these dangerous pathogens that are resistant to all classes of currently approved antibiotics. Resistance to even last-line therapy such as polymyxins (i.e., polymyxin B and colistin) has increasingly been reported, which virtually means no antibiotics will be available for treatment of PDR strains. Considering potential systemic toxicity and sub-optimal pharmacokinetic/pharmacodynamic (PK/PD) attainment, the topical use of antibiotics often remains a superior approach for the treatment and prophylaxis against PDR wound infections than parenteral administration, and direct pulmonary delivery of antibiotics via dry powder inhalation (DPI) holds great promise for treatment of PDR lung infections with minimal systemic exposure.

Teixobactins are a recently discovered novel antibiotic class that is the first and only resistance-resistant antibiotic; however, teixobactin typically possesses a narrow spectrum of activity against Gram-positive bacteria.

Advancement in the protection and/or medical treatment to address PDR infections, and broad spectrum, long-acting antibiotics is needed.

### SUMMARY OF THE INVENTION

The present invention is based, in part, on the development of broad-spectrum hybrid antibiotic molecules. Advantageously, these molecules can be resistant to multi-drug resistance (MDR) that may develop in pathogenic bacteria.

In an aspect, a teixobactin-antibiotic hybrid molecule comprising teixobactin covalently linked to a second, different antibiotic is provided. In some embodiments, the teixobactin is covalently linked to the antibiotic by a linker. In some embodiments, the antibiotic is a lipopeptide antibiotic, dalavancin, vancomycin, orivancin, telavancin, or a carbapenem. The lipopeptide antibiotic may be a cyclic lipopeptide antibiotic. In an embodiment, the lipopeptide antibiotic can be a polymyxin or derivative thereof, daptomycin, or an octapeptin, for example polymyxin B, a polymyxin derivative such as polymyxin B nonapeptide, FADDI-002 or FADD-115, or octapepin C4. In some embodiments, the hybrid molecules can dissociate into two component antibiotic molecules at an infection site.

The teixobactin and the antibiotic can be covalently linked via a linker. In an embodiment, the linker is a non-cleavable or non-labile linker. In an embodiment, the linker is a cleavable or labile linker. In some embodiments, the labile linker comprises a disulfide bond.

In an aspect, a pharmaceutical composition comprising the hybrid antibiotic molecule is provided, optionally comprising one or more excipients. In an aspect, the pharmaceutical composition is formulated for inhalation. In an aspect, the pharmaceutical composition is formulated as a dry powder for inhalation. In an aspect, the pharmaceutical composition is formulated as a hydrogel. In an aspect, the pharmaceutical is formulated for topical administration.

In another aspect, wound dressings comprising the pharmaceutical composition are provided.

One aspect of the invention is a method of treating a bacterial infection in a subject in need thereof, comprising administering to the subject a therapeutically effective amount of a hybrid antibiotic molecule or a pharmaceutical composition as described herein, thereby treating the bacterial infection in the subject.

In an aspect, the bacterial infection is a Gram-negative pathogen, for example, Pseudomonas aeruginosa, Acinetobacter baumannii, or Klebsiella pneumoniae. In an aspect, the bacterial infection is a Gram-positive pathogen, for example, methicillin-resistant Staphylococcus aureus (MRSA), vancomycin-resistant Staphylococcus aureus (VRSA), or Streptococcus pneumoniae. In some embodiments, the bacterial infection treated with the molecules or compositions is pan-drug resistant (PDR) or multi-drug resistant (MDR).

In an aspect, the administration is enteral, topical, or parenteral. The parenteral administration can be by inhalation or injection.

Another aspect of the invention is a method of treating a bacterial lung infection in a subject in need thereof, comprising administering to the subject a therapeutically effective amount of a hybrid antibiotic molecule or a pharmaceutical composition as described herein, thereby treating the bacterial lung infection in the subject. In an aspect, the molecule is administered by inhalation, e.g., as a dry powder composition, and can be administered via a dry powder inhaler. In an embodiment, the lung infection is pneumonia.

A further aspect of the invention is a method of treating a bacterial infection in a subject in need thereof, comprising administering to the subject a therapeutically effective amount of a topical composition comprising a hybrid antibiotic molecule or a pharmaceutical composition as described herein, thereby treating the bacterial infection in the subject. In an aspect, the infection is a wound infection, for example, a burn. In an aspect, the topical composition is a hydrogel. In an embodiment, the topical composition is provided on a solid substrate, e.g., a wound dressing.

The methods can comprise administration prior to identification of the bacterial infection as gram positive or gram negative. In an aspect, the bacteria do not develop resistance to the molecule or composition.

A further aspect of the invention is the use of a hybrid antibiotic molecule or a pharmaceutical composition as described herein for treating a bacterial infection in a subject, or the use of a hybrid antibiotic molecule or a pharmaceutical composition as described herein for the preparation of a medicament for treating a bacterial infection in a subject.

These and other aspects of the invention are set forth in more detail in the description of the invention below.

### BRIEF DESCRIPTION OF THE DRAWINGS

FIG. 1. Nuclear magnetic resonance (NMR) structure of teixobactin bound to the peptidoglycan building block lipid II (Left). Structure-Activity Relationship (SAR) model of the teixobactin-lipid II complex (Right).

FIGS. 2A-2B. Hydrogel-based localized release of colistin for antimicrobial treatment of burn wound infections. FIG. 2A. Synthesis of chitosan-colistin hydrogel. FIG. 2B. Schematic of exemplary on wound biodegradable hydrogel.

FIG. 3. Mouse burn infection model test of the colistin-loaded hydrogel against P. aeruginosa. Shown are blank infection control; blank HG-10 hydrogel; HG-10 with colistin (0.3 mg/wound); and colistin solution (0.3 mg/wound). Dashed line is detection limit.

FIG. 4. Octapeptin C4 lipopeptide SAR model (Left); SAR model of teixobactin-lipid II (Right).

FIGS. 5A-5C. Activity of dry powder inhalation (DPI) UOM-001 (teixobactin-polymyxin B hybrid) and -005 (texiobactin-FADDI-115 octapeptin hybrid) vs. individual compounds and their combinations in a rat lung infection model against P. aeruginosa FADDI-PA021m (FIG. 5A). Concentrations of the DPI formulations achieved in the epithelial lining fluid (ELF) (FIG. 5B). Percentage binding of teixobactin and UOM-001 to lung surfactant (FIG. 5C).

FIG. 6. Scanning electron microscope (SEM) images of the UOM-001 DPI.

FIGS. 7A-7B. UOM-001 does not display any toxicity on human A549 lung epithelial cells after 48 h of exposure (FIG. 7A). FIG. 7B. Lung histology in rat treated with UOM-001.

FIG. 8. Resistance-proof activity of the teixobactin-lipopeptide hybrid UOM-001 against P. aeruginosa FADDI-PA021m. No visible growth is seen after 48 h (far right).

### DETAILED DESCRIPTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art. In addition, any references cited herein are incorporated by reference in their entireties.

Unless otherwise defined, all technical and scientific terms used herein have the same meaning as commonly understood by one of skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Amino acids are represented herein in the manner recommended by the IUPAC-IUB Biochemical Nomenclature Commission, or (for amino acids) by either the one-letter code, or the three-letter code, both in accordance with 37 C.F.R. § 1.831 and established usage.

Except as otherwise indicated, standard methods known to those skilled in the art may be used for cloning genes, amplifying and detecting nucleic acids, and the like. Such techniques are known to those skilled in the art. See, e.g., Sambrook et al., Molecular Cloning: A Laboratory Manual 4th Ed. (Cold Spring Harbor, NY, 2012); Ausubel et al. Current Protocols in Molecular Biology (Green Publishing Associates, Inc. and John Wiley & Sons, Inc., New York).

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed singularly or in any combination.

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, ±5%, ±1%, ±0.5%, or even ±0.1% of the specified amount.

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” as used herein should not be interpreted as equivalent to ‘comprising.”

The term “consists essentially of” (and grammatical variants), as applied to a polypeptide or polynucleotide sequence of this invention, means a polypeptide or polynucleotide that consists of both the recited sequence (e.g., SEQ ID NO) and a total of ten or less (e.g., 1, 2, 3, 4, 5, 6, 7, 8, 9, or 10) additional amino acids on the N-terminal and/or C-terminal ends of the recited sequence or additional nucleotides on the 5′ and/or 3′ ends of the recited sequence such that the function of the polypeptide or polynucleotide is not materially altered. The total of ten or less additional amino acids or nucleotides includes the total number of additional amino acids or nucleotides on both ends added together. The term “materially altered,” as applied to polypeptides of the invention, refers to an increase or decrease in biological activities/properties (e.g., chaperone and/or isomerase activity) of at least about 50% or more as compared to the activity of a polypeptide consisting of the recited sequence.

The term “teixobactin” refers to the antimicrobial depsipeptide consisting of 11 amino acid residues with a characteristic cyclo-tetrapeptide substructure at the C-terminus and a methylated N-terminus. Derivatives of teixobactin comprise one or more modifications at any location on the teixobactin, e.g., at Position 1, 2, 3, 4, 5, 6, 7, 8, 9, 10 and/or 11, including substitutions as described herein. A derivative of teixobactin maintains at least 50% of the antibacterial activity of teixobactin, in some instances, at least 60%, 65%, 70%, 75%, 80%, 85%, 90%, 95%, 96%, 97%, 98%, 99%, or more antibacterial activity of teixobactin.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. In a broad aspect, the permissible substituents include acyclic and cyclic, branched and unbranched, carbocyclic and heterocyclic, aromatic and nonaromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Non-limiting examples of optional substituents as referred to herein include halogen, alkyl, aralkyl, heteroalkyl, alkenyl, alkynyl, cycloalkyl, hydroxyl, alkoxy, amino, amido, nitro, cyano, amido, carbonyl, carboxyl, silyl, ether, alkylthio, sulfonyl, sulfonamido, ketone, aldehyde, ester, heterocyclyl, aryl, and heteroaryl.

As used herein, the term “alkyl,” used either alone or in compound words such as “haloalkyl,” refers to an organic group that contains from 1 to 20 carbon atoms (i.e., C₁-C₂₀) including a straight-chain alkyl or branched alkyl, such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like.

The term “aryl” refers to an aromatic carbocyclic group of 6 to 20 carbon atoms having a single ring (e.g., phenyl) or multiple rings (e.g., biphenyl) or condensed multiple rings (e.g., naphthyl, fluorenyl). In some embodiments, aryl includes, but is not limited to, phenyl, fluorenyl, naphthyl, antryl and the like. Unless otherwise restricted by definition, an aryl may be optionally substituted by 1, 2, 3, 4 or 5 substituents (in some embodiments, 1, 2 or 3 substituents) as described herein.

The term “heteroaryl” refers to substituted or unsubstituted monocyclic, bicyclic, or polycyclic aromatic rings containing from 1 to 3 heteroatoms, such as N, O, or S, in the ring. “Heteroaryl” groups may be substituted with one or one or more substituents if so defined herein. Non-limiting examples of heteroaryl include, but are not limited to, furanyl, thienyl, pyrrolyl, pyrazolyl, imidazolyl, isoxazolyl, isothiazolyl, oxazolyl, 1,3-thiazolyl, triazolyl, oxadiazolyl, thiadiazolyl, tetrazolyl, pyridinyl, pyrimidinyl, pyrazinyl, pyridazinyl, triazinyl, indolyl, indazolyl, indolizinyl, isoindolyl, benzofuranyl, benzothienyl, benzoimidazolyl, 1,3-benzothiazolyl, 1,3-benzoxazolyl, purinyl, quinolinyl, isoquinolinyl, quinazolinyl, quinoxalinyl, 1,3-diazinyl, 1,2-diazinyl, 1,2-diazolyl, 1,4-diazanaphthalenyl, acridinyl, furo[3,2-b]pyridinyl, furo[3,2-c]pyridinyl, furo[2,3-c]pyridinyl, 6H-thieno[2,3-b]pyrrolyl, thieno[3,2-c]pyridinyl, thieno[2,3-d]pyrimidinyl, 1H-pyrrolo[2,3-b]pyridinyl, 1H-pyrrolo[2,3-c]pyridinyl, 1H-pyrrolo[3,2-b]pyridinyl, pyrrolo[1,2-a]pyrazinyl, pyrrolo[1,2-b]pyridazinyl, pyrazolo[1,5-a]pyridinyl, pyrazolo[1,5-a]pyrazinyl, imidazo[1,2-a]pyridinyl, 3H-imidazo[4,5-b]pyridinyl, imidazo[1,2-a]pyrimidinyl, imidazo[1,2-c]pyrimidinyl, imidazo[1,2-b]pyridazinyl, imidazo[1,2-a]pyrazinyl, imidazo[2,1-b][1,3]thiazolyl, imidazo[2,1-b][1,3,4]thiadiazolyl, [1,2,4]triazolo[1,5-a]pyridinyl, [1,2,4]triazolo[4,3-a]pyridinyl and the like. A heteroaryl radical may optionally be substituted on a carbon or nitrogen atom ring member with substituent species as described herein where allowed by available valences.

As used herein, the term “halo,” “halogen” or “halogenated” refers to a halogen atom radical, including fluoro, chloro, bromo and iodo.

“C6 to C8 fatty acyl” refers to an acyl group derived from fatty acids having 6 to 8 carbon atoms and includes saturated and unsaturated fatty acids.

The term “aromatic moiety,” as used herein, refers to an aryl that may be a mono- or polycyclic, unsaturated moiety having 3 to 14 carbon atoms, each of which may be substituted or unsubstituted.

A “lipidic aromatic group” refers to a lipid soluble aromatic group. In some embodiments, the lipidic aromatic group is unsubstituted.

The term “linker” refers to a chemical entity which connects a teixobactin molecule to an antibiotic molecule. A linker may be a non-labile linker (non-cleavable) or a labile linker (cleavable linker). The labile linker allows the antibiotic molecule to be cleaved from the teixobactin molecule in order to provide a maximal effect in the cell. These labile linkers include hydrolytically labile (acid labile) linkers, reductively labile linkers (principally disulfide linkers which are reductively cleaved by intracellular glutathione or other disulfide reducing agent) and enzymatically labile linkers (protease substrates).

As used herein, “unsaturated” refers to compounds or structures having at least one degree of unsaturation (e.g., at least one double or triple bond).

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring can also be designated as “cis” or “trans.” The term “cis” represents substituents on the same side of the plane of the ring, and the term “trans” represents substituents on opposite sides of the plane of the ring. Mixtures of compounds wherein the substituents are disposed on both the same and opposite sides of plane of the ring are designated “cis/trans.”

All chiral, diastereomeric, racemic, and geometric isomeric forms of a structure are intended, unless specific stereochemistry or isomeric form is specifically indicated. All processes used to prepare compounds and intermediates made therein are encompassed by the present disclosure. All tautomers of shown or described compounds are also encompassed by the present disclosure.

When any variable (e.g., Ri) occurs more than one time in any constituent or formula for a compound, its definition at each occurrence is independent of its definition at every other occurrence. Thus, for example, if a group is shown to be substituted with one or more Ri moieties, then Ri at each occurrence is selected independently from the Markush group recited for Ri. Also, combinations of substituents and/or variables are permissible, but only if such combinations result in stable compounds within a designated atom's normal valency.

“Pharmaceutically acceptable carrier” (sometimes referred to as a “carrier”) refers to a carrier or excipient that is useful in preparing a pharmaceutical or therapeutic composition that is generally safe and non-toxic and includes a carrier that is acceptable for veterinary and/or human pharmaceutical or therapeutic use. The term “carrier” or “pharmaceutically acceptable carrier” can include, but is not limited to, phosphate-buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well-known in the art for use in pharmaceutical formulations and as described further herein.

The term “modulate,” “modulates,” or “modulation” refers to enhancement (e.g., an increase) or inhibition (e.g., a decrease) in the specified level or activity.

The term “enhance” or “increase” refers to an increase in the specified parameter of at least about 1.25-fold, 1.5-fold, 2-fold, 3-fold, 4-fold, 5-fold, 6-fold, 8-fold, 10-fold, 12-fold, or even 15-fold and/or can be expressed in the enhancement and/or increase of a specified level and/or activity of at least about 1%, 5%, 10%, 15%, 25%, 35%, 40%, 50%, 60%, 75%, 80%, 90%, 95% or more.

“Inhibit” or “reduce” or grammatical variations thereof as used herein refers to a decrease or diminishment in the specified level or activity of at least about 1, 5, 10, 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

The term “contact” or grammatical variations thereof, as used with respect to a hybrid molecule and a cell, refers to bringing the hybrid molecule and the cell in sufficiently close proximity to each other for one to exert a biological effect on the other.

A “subject” may be any vertebrate organism in various embodiments. A subject may be an individual to whom an agent is administered, e.g., for experimental, diagnostic, and/or therapeutic purposes or from whom a sample is obtained or on whom a procedure is performed. In some embodiments, a subject is a mammal, e.g., a human, non-human primate, lagomorph (e.g., rabbit), or rodent (e.g., mouse, rat). In some embodiments, a human subject is a neonate, child, adult, or geriatric subject.

“Treat,” “treating,” and similar terms as used herein, in the context of treating a subject, refer to providing medical and/or surgical management of a subject. Treatment may include, but is not limited to, administering an agent or composition (e.g., a pharmaceutical composition) to a subject. Treatment is typically undertaken in an effort to alter the course of a disease (which term is used to indicate any disease, disorder, syndrome, or undesirable condition warranting or potentially warranting therapy) in a manner beneficial to the subject. The effect of treatment may include reversing, alleviating, reducing severity of, curing, inhibiting the progression of, and/or reducing the likelihood of occurrence or recurrence of the disease or one or more symptoms or manifestations of the disease. A therapeutic agent may be administered to a subject who has a disease or is at increased risk of developing a disease relative to a member of the general population. In some embodiments, a therapeutic agent may be administered to a subject who has had a disease but no longer shows evidence of the disease. The agent may be administered e.g., to reduce the likelihood of recurrence of evident disease. A therapeutic agent may be administered prophylactically, i.e., before development of any symptom or manifestation of a disease. “Prophylactic treatment” refers to providing medical and/or surgical management to a subject who has not developed a disease or does not show evidence of a disease in order, e.g., to reduce the likelihood that the disease will occur, delay the onset of the disease, or to reduce the severity of the disease should it occur. The subject may have been identified as being at risk of developing the disease (e.g., at increased risk relative to the general population or as having a risk factor that increases the likelihood of developing the disease.

Grammatical variations of “administer,” “administration,” and “administering” to a subject include any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration,” “administration in combination,” “simultaneous administration,” or “administered simultaneously” as used herein, means that the compounds are administered at the same point in time, overlapping in time, or one following the other. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g., greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

The present invention is based in part on Applicant's development of hybrid molecules that overcome the impermeability of the Gram-negative outer membrane toward teixobactin by coupling polymyxin-like lipopeptides onto the teixobactin molecule via a labile linker to generate a broad-spectrum hybrid molecule that dissociates into the two component molecules in vivo at the infection site. The novel teixobactin-lipopeptide hybrids are superior to native teixobactin as they retain the key anti-resistance property of teixobactin and, in addition, have a broader-spectrum, with potent activity against MDR Gram-negative bacteria, as well as MDR Gram-positive bacteria.

### Teixobactin Hybrid Antibiotic Molecules and Compositions

Teixobactin-antibiotic hybrid molecules are provided, comprising teixobactin covalently linked to a second, different antibiotic. The teixobactin may be covalently linked to the antibiotic by a linker.

In an embodiment, the teixobactin used in the hybrid antibiotic molecule is unmodified and has the following structure:

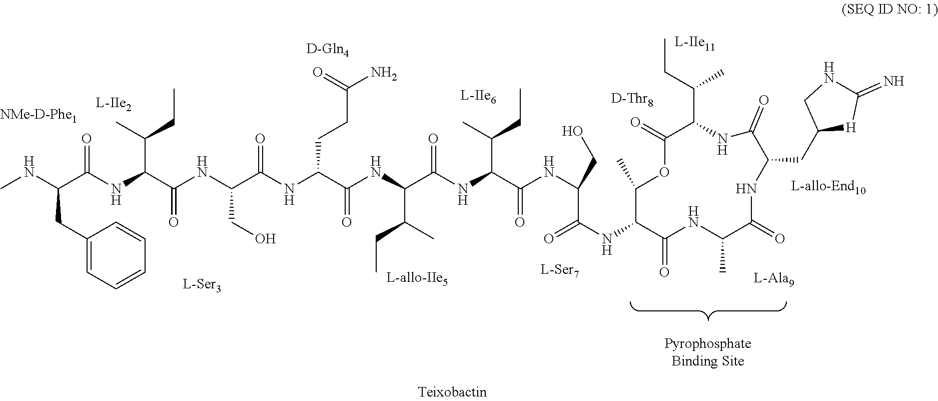

In an embodiment, the teixobactin can have one or more modifications as described in Table 5. In some embodiments, the modifications can comprise lipidic modifications at the N-terminus at Positions 1, 2, and/or 3. In an embodiment, the modifications comprise (1) combinations of C6 to C8 fatty acyl, heteroaryl, halogenated, and/or lipidic aromatic groups; (2) various alternate alkyl (branched, unsaturated) and/or aromatic moieties; and/or (3) lipidic amino acids. In one embodiment, the modification provides substantially stronger hydrophobic interactions with the prenyl chain of lipid II and enhances Gram-negative outer membrane anchoring/penetration. In some embodiments, the chemical modification comprises mixed lipidic and polar modifications at the N-terminus at Positions 1, 2, and/or 3. In an embodiment, the modifications comprise any combination of the lipidic modifications above with: (1) heteroaryl, halogenated aromatic groups (2) one or more H-bonding residues (Thr, Ser), (3) alternate basic residues (Lys, Orn, Arg, His), acidic residues (Glu, Asp), or (4) H-bonding residues (Asn, Gln). In an embodiment, the modifications optimize the Gram-negative outer membrane anchoring/penetration, which is known to require polar interactions with lipid A and other outer membrane phospholipids.

The antibiotic can be, without limitation, a lipopeptide antibiotic, dalavancin, vancomycin, orivancin, telavancin, or a carbapenem. In an aspect, the lipopeptide antibiotic is a cyclic lipopeptide antibiotic. Example lipopeptide antibiotics include a polymyxin or derivative thereof, daptomycin, or an octapeptin. In an embodiment, the antibiotic in the hybrid molecule enhances Gram-negative outer membrane permeabilizing activity thereby allowing higher concentrations of the teixobactin to reach its intracellular lipid II target.

In an aspect, the polymyxin is polymyxin B or colistin (polymyxin E). In an embodiment, the polymyxin derivative is polymyxin B nonapeptide, FADDI-002, or FADDI-115.

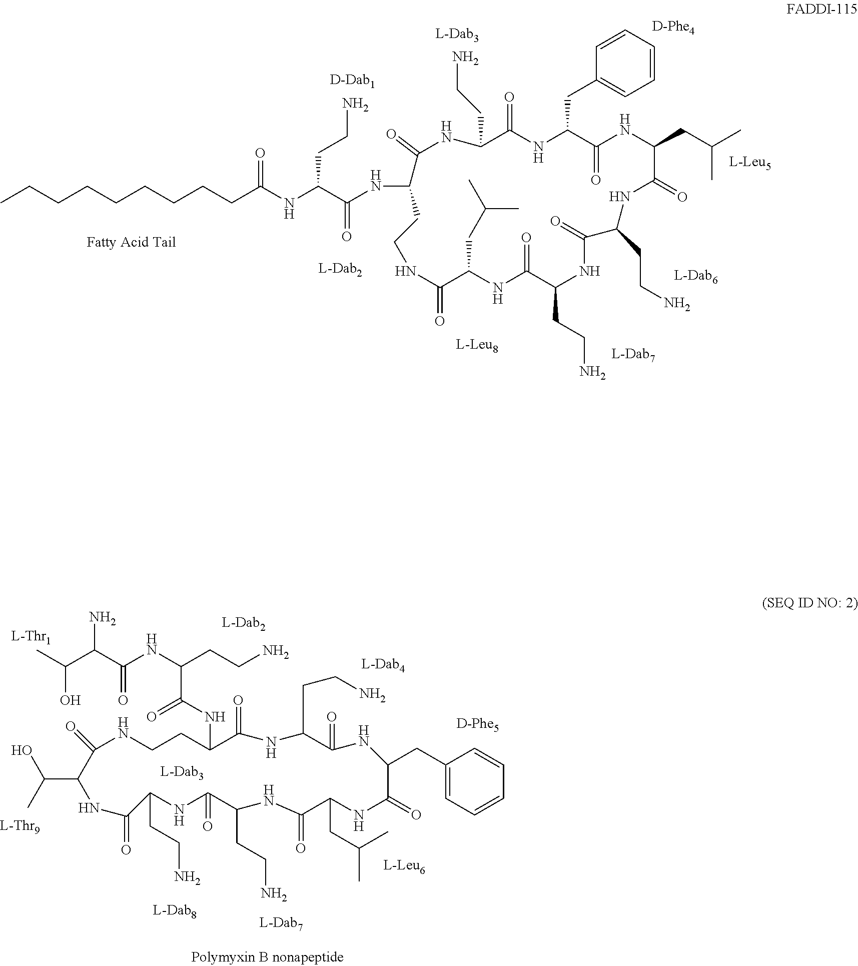

Example polymyxin derivatives include, but are not limited to, the derivatives in Table 1, as described in Velkov et al. (2014) ACS Chem. Biol. 9(5):1172-1177, incorporated herein by reference in its entirety.

TABLE 1
Polymyxin derivatives.

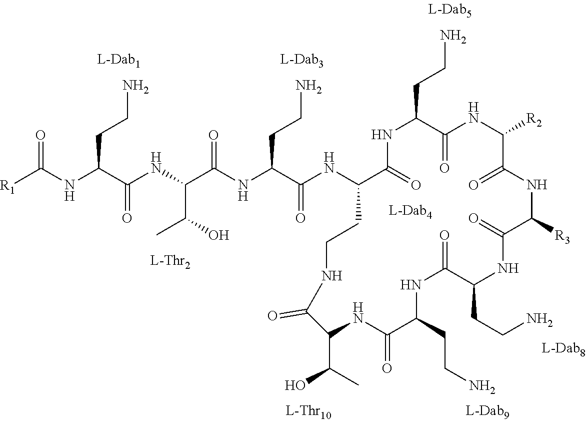

| Derivative | R₁ | R₂ | R₃ |
|---|---|---|---|
| FADDI-002 | CH₃(CH₂)₆- (octanoyl) | 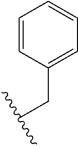 [SMILES: *Cc1ccccc1] | —(CH₂)₇CH₃ (L-OctylGly) |
| | | (D-Phe) | |
| FADDI-003 | 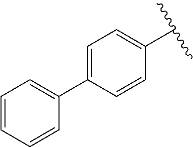 [SMILES: *c1ccc(-c2ccccc2)cc1] | 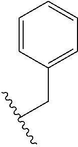 [SMILES: *Cc1ccccc1] | —(CH₂)₇CH₃ (L-OctylGly) |
| | (biphenylacyl) | (D-Phe) | |
| FADDI-019 | CH₃(CH₂)₆- (octanoyl) | —(CH₂)₇CH₃ (L-OctylGly) | 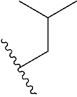 [SMILES: *CC(C)C] |
| | | | (L-Leu) |
| FADDI-287 | CH₃(CH₂)₆- (octanoyl) | 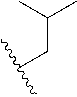 [SMILES: *CC(C)C] | —CH₂CH₃ (L-Abu) |
| | | (L-Leu) | |

Exemplary octapepins are described in, e.g., Velkov et al. (2018) Cell Chem Biol. 25(4):380-391.e5, incorporated herein by reference in its entirety. In an embodiment, the octapeptin is octapeptin C4:

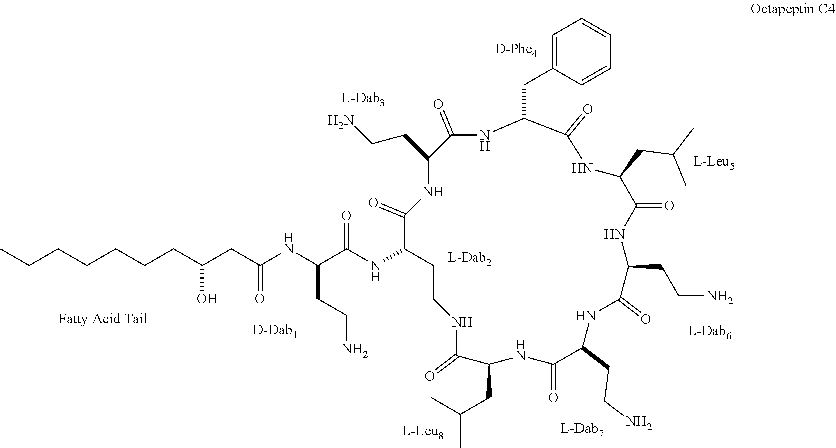

The hybrid molecule may comprise a linker covalently linking the teixobactin and antibiotic. In an aspect, the linker is a non-cleavable or non-labile linker. In an aspect, the linker is a cleavable or labile linker. In some embodiments, the labile linker comprises a disulfide bond. Linkers are known in the art, including those for controlled release in hydrogels, see, e.g., WO 2012/0396602, incorporated herein by reference.

Compositions comprising the compounds are also provided herein. In an embodiment, a pharmaceutical composition is provided comprising a hybrid molecule as described herein and optionally comprising one or more excipients. The pharmaceutical composition can be formulated for inhalation, for example, as a dry powder for inhalation.

In some embodiments, the compound is:

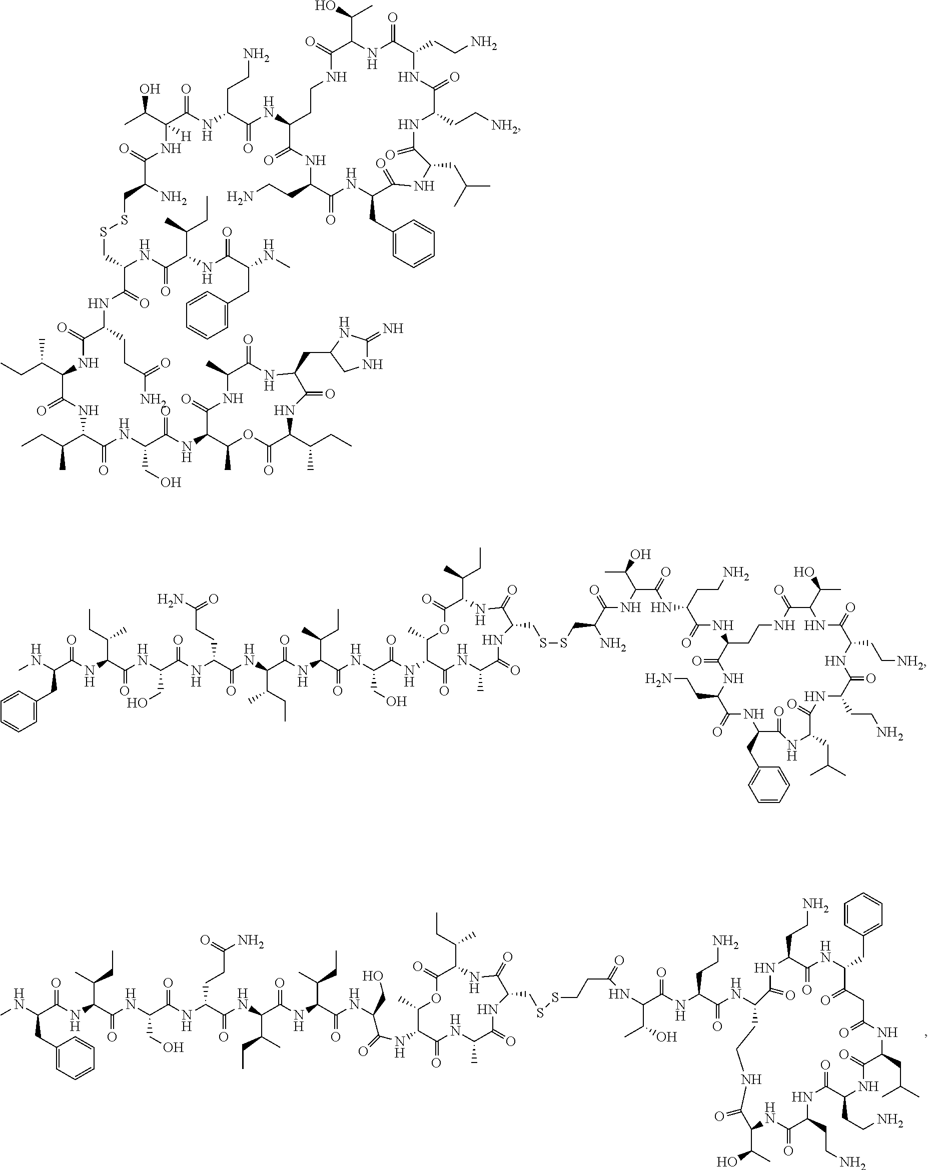

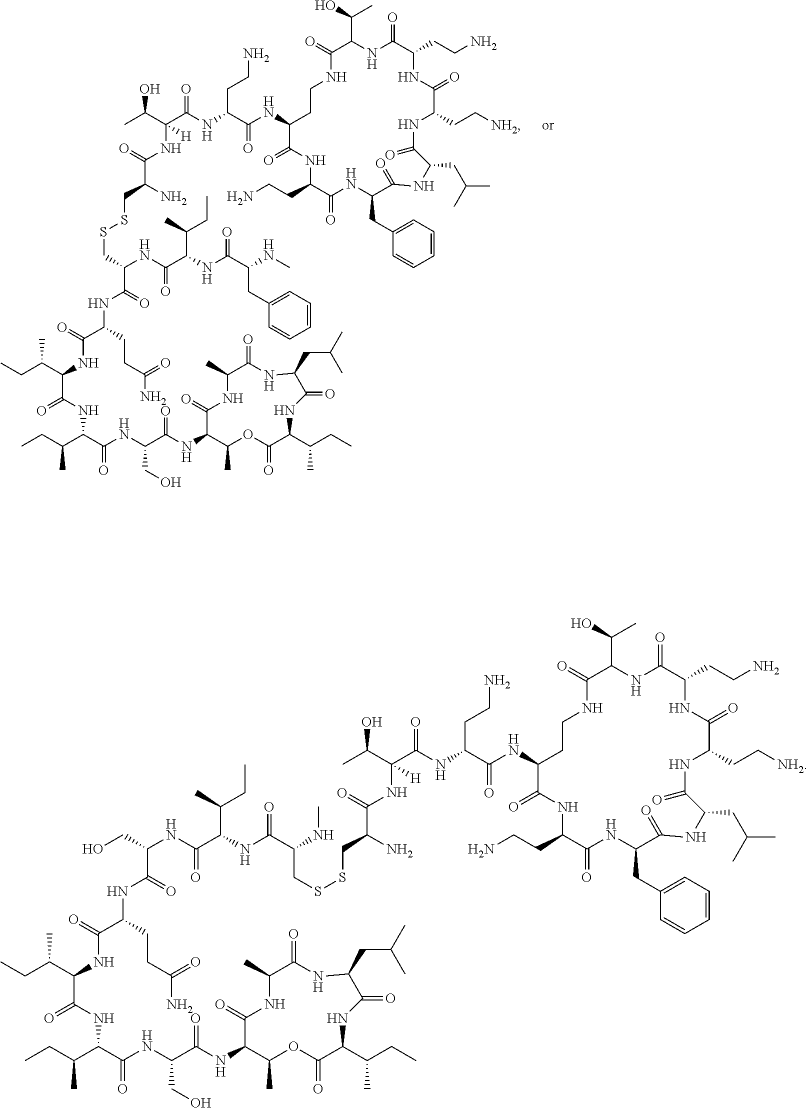

Compositions comprising the hybrid molecules are also provided herein. In an embodiment, a pharmaceutical composition is provided comprising at least one hybrid molecule (e.g., 1, 2, 3, 4, 5 or more hybrid molecules) as described herein and optionally comprising one or more excipients. The pharmaceutical composition can be formulated for inhalation, for example, as a dry powder for inhalation.

In some embodiments, the pharmaceutical composition is formulated for topical application, for example as a hydrogel. A solid substrate, such as a wound dressing or bandage comprising the pharmaceutical composition can also be provided.

As a further aspect, the invention provides pharmaceutical formulations and methods of administering the same to achieve any of the therapeutic effects (e.g., treatment of a bacterial infection, e.g., a MDR infection) discussed above. The pharmaceutical formulation may comprise any of the reagents discussed above in a pharmaceutically acceptable carrier. By “pharmaceutically acceptable” it is meant a material that is not biologically or otherwise undesirable, i.e., the material can be administered to a subject without causing any undesirable biological effects such as toxicity. The formulations of the invention can optionally comprise medicinal agents, pharmaceutical agents, carriers, adjuvants, dispersing agents, diluents, and the like.

One embodiment of the invention is a composition comprising a hybrid molecule as described herein and a suitable carrier, diluent, or excipient, and optionally a pharmaceutically acceptable carrier, diluent, or excipient. In one embodiment, the composition is in a form suitable for parenteral, oral, rectal, systemic, urogenital, topical, inhalation, intravitreal, intraocular, otic, intranasal, dermal, sublingual, or buccal administration.

The hybrid molecule of the invention can be formulated for administration in a pharmaceutical carrier in accordance with known techniques. See, e.g., Remington, The Science and Practice of Pharmacy (23rd Ed. 2020). In the manufacture of a pharmaceutical formulation according to the invention, the hybrid antibiotic molecule (including the physiologically acceptable salts thereof) is typically admixed with, inter alia, an acceptable carrier. The carrier can be a solid or a liquid, or both. One or more hybrid molecules can be incorporated in the formulations of the invention, which can be prepared by any of the well-known techniques of pharmacy.

A further aspect of the invention is a method of treating subjects in vivo, comprising administering to a subject a pharmaceutical composition comprising a hybrid molecule of the invention in a pharmaceutically acceptable carrier, wherein the pharmaceutical composition is administered in a therapeutically effective amount. Administration of the hybrid molecule of the present invention to a human subject or an animal in need thereof can be by any means known in the art for administering compounds.

Non-limiting examples of formulations of the invention include those suitable for oral, rectal, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intracranial, intrathecal, and inhalation administration, administration to the liver by intraportal delivery, as well as direct organ injection (e.g., into the liver, into a limb, into the brain or spinal cord for delivery to the central nervous system, into the pancreas, or into a tumor or the tissue surrounding a tumor). The most suitable route in any given case will depend on the nature and severity of the condition being treated and on the nature of the particular compound, which is being used, e.g., treatment of lung infections with a dry powder formulation of the hybrid molecule. In an embodiment, administration may be topical delivery by a hydrogel formulation. In some embodiments, it may be desirable to deliver the formulation locally to avoid any side effects associated with systemic administration. For example, local administration can be accomplished by direct injection at the desired treatment site, by introduction intravenously at a site near a desired treatment site (e.g., into a vessel that feeds a treatment site, or intramuscular administration). In certain embodiments, the formulation can be a slow release formulation, e.g., in the form of a slow release depot.

For injection, the carrier will typically be a liquid, such as sterile pyrogen-free water, pyrogen-free phosphate-buffered saline solution, bacteriostatic water, or Cremophor EL® (BASF, Parsippany, NJ). For other methods of administration, the carrier can be either solid or liquid.

For oral administration, the compound can be administered in solid dosage forms, such as capsules, tablets, and powders, or in liquid dosage forms, such as elixirs, syrups, and suspensions. Compounds can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for buccal (sub-lingual) administration include lozenges comprising the compound in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the compound, which preparations are preferably isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose containers, for example sealed ampoules and vials, and can be stored in a freeze-dried (lyophilized) condition requiring only the addition of the sterile liquid carrier, for example, saline or water-for-injection immediately prior to use. Extemporaneous injection solutions and suspensions can be prepared from sterile powders, granules and tablets of the kind previously described. For example, in one aspect of the present invention, there is provided an injectable, stable, sterile composition comprising a compound of the invention, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is pharmaceutically acceptable can be employed in sufficient quantity to emulsify the compound or salt in an aqueous carrier. One such useful emulsifying agent is phosphatidyl choline.

Formulations suitable for rectal administration are preferably presented as unit dose suppositories. These can be prepared by admixing the compound with one or more conventional solid carriers, for example, cocoa butter, and then shaping the resulting mixture.

Formulations suitable for topical application to the skin preferably take the form of an hydrogel, ointment, cream, lotion, paste, gel, spray, aerosol, or oil. Carriers which can be used include petroleum jelly, lanolin, polyethylene glycols, alcohols, transdermal enhancers, and combinations of two or more thereof. In example embodiments, hydrogel formulations as described in the working examples are used for treatment of topical infections.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Tyle (1986) Pharm. Res. 3:318) and typically take the form of an optionally buffered aqueous solution of the hybrid molecule. Suitable formulations comprise citrate or bis tris buffer (pH 6) or ethanol/water and contain from 0.1M to 0.2M of the hybrid molecule.

The hybrid molecule can alternatively be formulated for nasal administration or otherwise administered to the lungs of a subject by any suitable means, e.g., administered by an aerosol suspension of respirable particles comprising the hybrid molecule, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones (1987) Drug Delivery to the Respiratory Tract, Ellis Horwood; Gonda (1990) Critical Reviews in Therapeutic Drug Carrier Systems 6:273-313; and Raeburn et al. (1992) J. Pharmacol. Toxicol. Meth. 27:143. Aerosols of liquid particles comprising the hybrid molecule can be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the hybrid molecule can likewise be produced with any solid particulate medicament aerosol generator, by techniques known in the pharmaceutical art. Alternatively, one can administer the hybrid molecule in a local rather than systemic manner, for example, in a depot or sustained-release formulation.

Further, the present invention provides liposomal formulations of the hybrid molecules disclosed herein and salts thereof. The technology for forming liposomal suspensions is well-known in the art. When the hybrid molecule or salt thereof is an aqueous-soluble salt, using conventional liposome technology, the same can be incorporated into lipid vesicles. In such an instance, due to the water solubility of the com hybrid molecule pound or salt, the hybrid molecule or salt will be substantially entrained within the hydrophilic center or core of the liposomes. The lipid layer employed can be of any conventional composition and can either contain cholesterol or can be cholesterol-free. When the hybrid molecule or salt of interest is water-insoluble, again employing conventional liposome formation technology, the hybrid molecule or salt can be substantially entrained within the hydrophobic lipid bilayer which forms the structure of the liposome. In either instance, the liposomes which are produced can be reduced in size, as through the use of standard sonication and homogenization techniques.

The liposomal formulations containing the hybrid molecules disclosed herein or salts thereof, can be lyophilized to produce a lyophilizate which can be reconstituted with a pharmaceutically acceptable carrier, such as water, to regenerate a liposomal suspension.

In the case of water-insoluble molecules, a pharmaceutical composition can be prepared containing the water-insoluble molecule, such as for example, in an aqueous base emulsion. In such an instance, the composition will contain a sufficient amount of pharmaceutically acceptable emulsifying agent to emulsify the desired amount of the molecule. Particularly useful emulsifying agents include phosphatidylcholines and lecithin.

### Methods

Methods of treating a bacterial infection in a subject in need thereof are provided, comprising administering to the subject a therapeutically effective amount of at least one hybrid molecule (e.g., 1, 2, 3, 4, 5 or more hybrid molecules) or a pharmaceutical composition comprising the hybrid molecule, thereby treating the bacterial infection in the subject. Subjects in need of treatment with a hybrid molecule or a pharmaceutical composition comprising the hybrid molecule as described herein include those having, suspected of having, or at risk of having a bacterial infection. A subject having or suspected of having a bacterial infection may exhibit one or more signs or symptoms of an infection, e.g., fever, chills, sweats, change in cough or a new cough, sore throat or new mouth sore, shortness of breath, nasal congestion, stiff neck, burning or pain with urination, unusual vaginal discharge or irritation, increased urination, redness or swelling in any area including surgical wounds and ports, diarrhea, vomiting, pain in the abdomen or rectum, skin rash, fatigue, swollen lymph nodes, and/or new onset of pain. A subject at risk of having a bacterial infection may include a wounded subject or a subject who has undergone surgery. In some embodiments, the hybrid molecule is administered to a compromised subject, e.g., a subject with an immune disorder or other disease such as chronic obstructive pulmonary disease, cystic fibrosis, or bronchiectasis, which renders the subject susceptible to bacterial infection. In some embodiments, the hybrid molecule is administered to a wound or burn, which is at risk of bacterial infection. In some embodiments, the wound is an open wound. The open wound may be a gunshot wound, a puncture wound, a laceration wound, an abrasion, a cut, a penetrating wound, a surgical wound, or any other wound. In some embodiments, the wound may be a puncture wound, e.g., a puncture wound resulting from hemodialysis or catheterization.

Advantageously, in some embodiments, the methods are effective in treating a bacterial infection that is pan-drug resistant (PDR) or multi-drug resistant (MDR). In some embodiments, the bacterial infection is a Gram-negative pathogen. In some embodiments, the Gram-negative pathogen is Pseudomonas aeruginosa, Acinetobacter baumannii, or Klebsiella pneumoniae. In some embodiments, the bacterial infection is a Gram-positive pathogen. In some embodiments, the Gram-positive pathogen is methicillin-resistant Staphylococcus aureus (MRSA), vancomycin-resistant Staphylococcus aureus (VRSA), or Streptococcus pneumoniae.

The amount of the disclosed compositions administered to a subject will vary from subject to subject, depending on the nature of the disclosed compositions and/or formulations, the species, gender, age, weight and general condition of the subject, the mode of administration, and the like. Effective dosages and schedules for administering the compositions may be determined empirically, and making such determinations is within the skill in the art. The dosage ranges for the administration of the disclosed compositions are those large enough to produce the desired effect (e.g., to treat a bacterial infection or to treat multi-drug resistant bacteria). The dosage should not be so large as to outweigh benefits by causing extensive or severe adverse side effects, such as unwanted cross-reactions, anaphylactic reactions, and the like, although some adverse side effects may be expected. The dosage can be adjusted by the individual clinician in the event of any counterindications. Generally, the disclosed compositions and/or formulations are administered to the subject at a dosage of active component(s) ranging from 0.01 mg/kg body weight to 100 g/kg body weight. In some embodiments, the disclosed compositions and/or formulations are administered to the subject at a dosage of active component(s) ranging from 0.01 mg/kg to 10 g/kg, 0.1 mg/kg to 10 g/kg, 1 mg/kg to 10 g/kg, from 10 mg/kg to 1 g/kg, from 10 mg/kg to 500 mg/kg, from 10 mg/kg to 100 mg/kg, from 10 mg/kg to 10 mg/kg, from 10 mg/kg to 1 mg/kg, from 10 mg/kg to 500 mg/kg, or from 10 mg/kg to 100 mg/kg body weight. Dosages above or below the range cited above may be administered to the individual subject if desired. The compositions can be administered in any herein disclosed pharmaceutical composition comprising a pharmaceutically acceptable carrier.

Methods of treating a bacterial lung infection in a subject in need thereof are provided, comprising administering to the subject a therapeutically effective amount of the hybrid molecule, thereby treating the bacterial lung infection in the subject. In some embodiments, the administration is by inhalation or injection. In an embodiment, the molecule is administered as a dry powder composition. In some embodiments, the dry powder composition is administered via a dry powder inhaler. In an embodiment, the lung infection is pneumonia.

Methods of treating a bacterial infection in a subject in need thereof are provided, comprising administering to the subject a therapeutically effective amount of a topical composition comprising a hybrid molecule as described herein, thereby treating the bacterial infection in the subject. In an embodiment, the infection is a wound infection. In some embodiments, the wound is a burn. The topical composition comprising the hybrid molecule can be a hydrogel. In some embodiments, the topical composition is provided on a solid substrate, e.g., a wound dressing. In an embodiment, administration is enteral, topical, or parenteral.

In embodiments, the administration occurs prior to identification of the bacterial infection as Gram-positive or Gram-negative. In embodiments, the bacteria do not develop resistance to the molecule or composition.

Having described the present invention, the same will be explained in greater detail in the following examples, which are included herein for illustration purposes only, and which are not intended to be limiting to the invention.

### EXAMPLES

### Example 1. Hydrogel

Teixobactin has a unique mode of action that entails disrupting bacterial cell wall synthesis through binding and inhibition of lipid II, which is crucial for the biosynthesis of the peptidoglycan structural component of the cell wall, thus leading to cell death via autolysis (Ling et al. (2015) Nature 517:455-459; Homma et al. (2016) Antimicrob. Agents Chemother. 60:6510-6517). Furthermore, the lipid II structure is unique within the bacterial kingdom, thereby preventing possible toxic effects of lipid II binders on mammalian cells (Liu et al. (2017) Sci. Rep. 7:17197). This is possibly why it remains effective against vancomycin-resistant strains of bacteria and there is no cross-resistance (Ling et al. (2015) Nature 517:455-459). Presumably, native teixobactin is inactive against Gram-negative bacteria as it cannot permeate the highly impermeable outer membrane. With no outer membrane present in Gram-positive bacteria, the cell wall is readily accessible by antibiotics. As described herein, the novel teixobactin-lipopeptide conjugates explored in these examples have broad-spectrum activity against both Gram-negative and Gram-positive bacteria without any emergence of resistance. This example explores the development of the first resistance-proof, broad spectrum, long-acting teixobactin conjugated or combined with polymyxin lipopeptide potentiators in a hydrogel formulation for delivery against wound infections. This hydrogel dressing is expected to be stable, durable, and suitable for field deployment, representing a major break-through for the treatment of PDR wound infections, e.g., in combat personnel.

Optimized Chemical Synthesis of Teixobactins. Applicant devised a convergent synthetic strategy, which can achieve preparation of large quantities of both the N-terminal linear protected fragment and the C-terminal macrocycle. It is thus only necessary to synthesize modifications of these shorter precursors to generate new teixobactin analogs (rather than performing a full stepwise assembly). Accordingly, preparation of the proposed 150 analogues for lead optimization below is expedited. This convergent synthetic 7+4 approach, which involves assembly of the protected N-terminal fragment and the macrocyclic core is depicted in Scheme 1. Using this scheme, 88 novel teixobactins were prepared with purity >95%. Furthermore, scale-up syntheses provided >100 mg of key teixobactins for in vitro and in vivo evaluations.

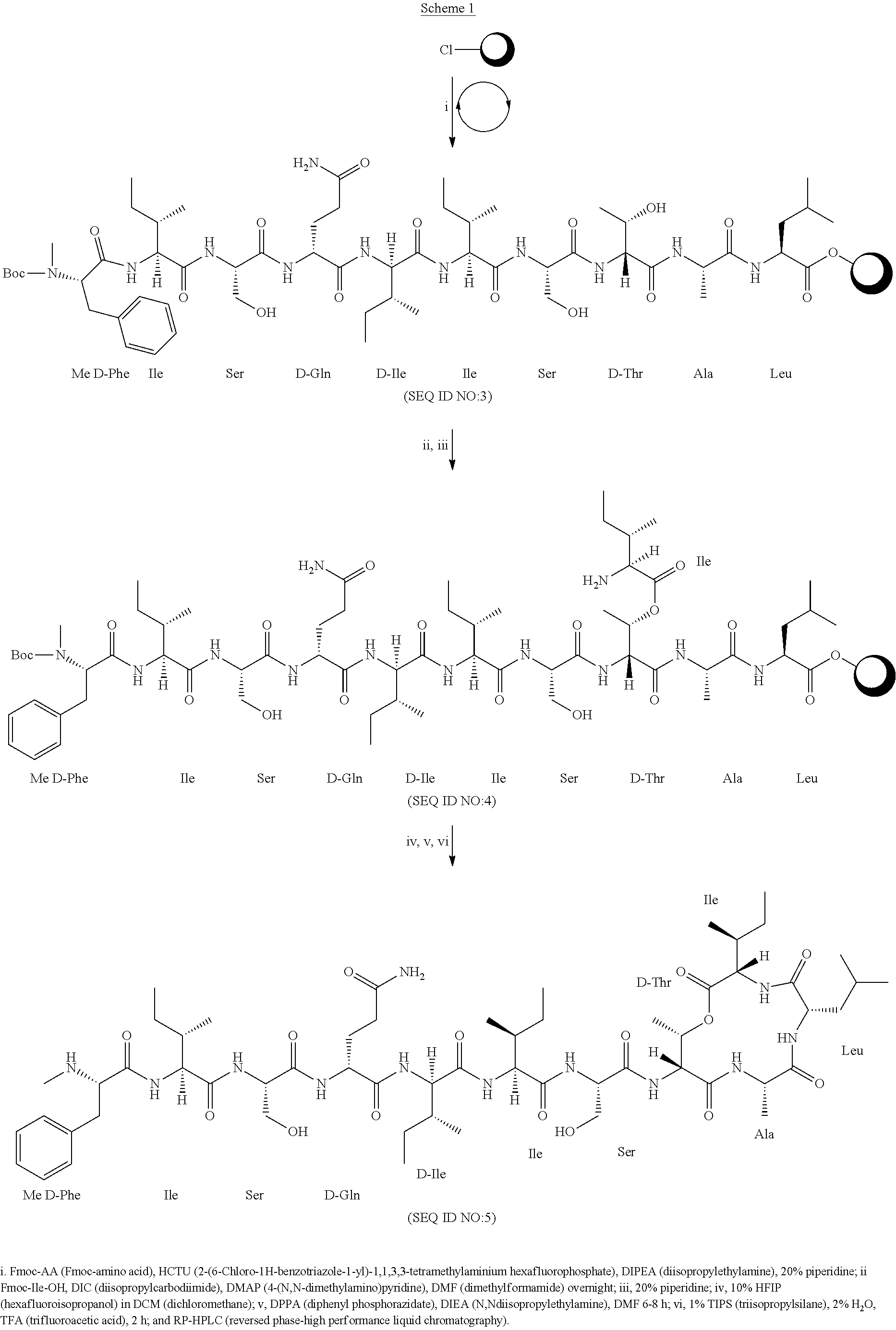

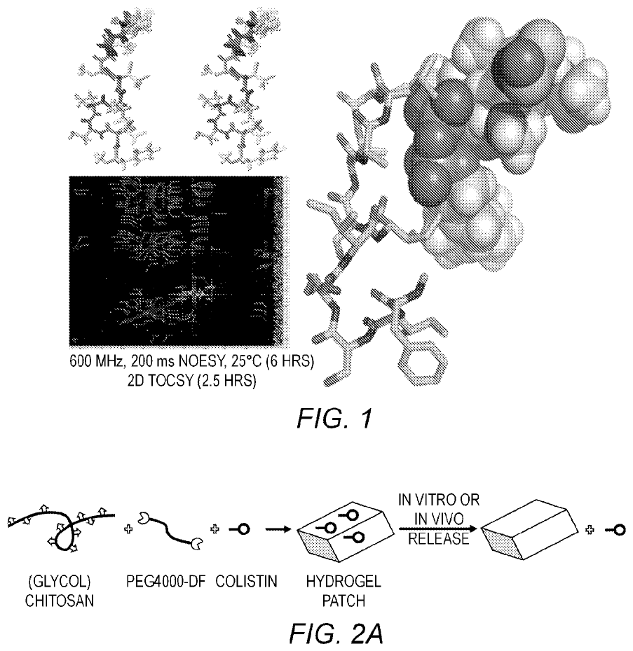

Discovery of Novel Broad-Spectrum Teixobactins and Conjugates with Polymyxin-Like Lipopeptide. The precise mechanism of interaction of teixobactin with its primary target, lipid II, is unknown, but what is known is its mode of action may involve self-assembly into amyloid-like aggregates (Liu et al. (2017) Sci. Rep. 7:17197; Wen et al. (2018) Chem Sci. 9:6997-7008; Wen et al. (2018) Biophysical J. 114: 456a; Yang et al. (2017) Chem. Comm. (Cambridge, England) 53:2772-2775; Yang et al. (2018) J. Am. Chem. Soc. 140:14028-14032; Öster et al. (2018) Chem. Sci. 9:8850-8859). The lack of structure-conformation relationships understanding of teixobactin interactions with its lipid II target and bacterial membrane lipids is a major knowledge-gap limiting the progression of the field. Transferred nuclear Overhauser effect (trNOE) is a highly sensitive NMR method to examine the bound conformation of peptides. It has been demonstrated that small (i.e., sub-stoichiometric) quantities of the paramagnetic lanthanide shift reagent thulium-complexed dipicolinic acid, [Tm(DPA)₃]³—, can differentially alter the chemical shift signature of the signals in the free spectra of teixobactin leading to complementary trNOE data sets. From three unique sets of 2D trNOESY data (with two sets measured in the presence of different amounts of the shift reagent) a high-resolution trNOE structure of native teixobactin in dodecylphosphocholine (DPC) micelles and in the presence of a synthetic lipid II was calculated (FIG. 1) (Schwartz et al. (2001) J. Am. Chem. Soc. 123:11638-11643). This allowed us to construct a SAR model for the teixobactin-lipid II complex for rational teixobactin design. The SAR model revealed that the binding of teixobactin to lipid II involves two fundamental interactions: (1) An initial interaction between the teixobactin macrocycle (which forms a cage-like structure) with the lipid II phosphates. (2) A stabilizing hydrophobic interaction between the N-terminal residues 1-3 of teixobactin and the hydrophobic prenyl chain of lipid II (FIG. 1). To refine the SAR model, a set of 10 teixobactin conformational probes was synthesized using regio-specific stereo-chemical positional scanning and repeated the NMR structural studies to discover the contribution of individual residues to the lipid II binding. This refined SAR model confirmed that the hydrophobic contribution from the N-terminal residues 1-3 is the predominant driving force for lipid II complexation following the initial binding event of the cage-like macrocycle with the lipid II phosphates. The contemporary understanding of the physicochemical factors that dictate a molecule's ability to cross the Gram-negative bacterial outer membrane indicate the necessity for mixed polar and hydrophobic characteristics (Nikaido (2003) MMBR 67:593-656). Bearing these principles in mind with the SAR model, a series of first generation broad-spectrum teixobactins (e.g., UOM-001) were designed with mixed polar/hydrophobic heteroaryl aromatic groups (e.g., pryridyl, benzoimidazole, thiophene, furan) substituted at positions 1-3 of the N-terminus (Table 2) and additional substituents substituted at positions 3, 4 and 7 (Table 3).

TABLE 2
Broad-spectrum teixobactins with mixed polar/hydrophobic heteroaryl aromatic
groups substituted at positions 1-3 of the N-terminus of teixobactin (SEQ ID NO: 1).

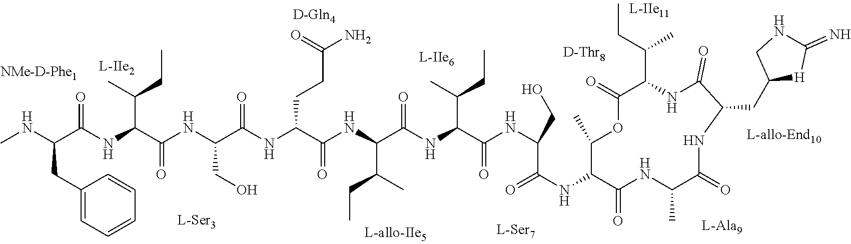

(SEQ ID NO: 1)
Heteroaryl aromatic Groups

| | | |
|---|---|---|
| 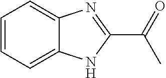 | 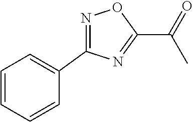 | 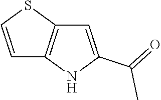 |
| 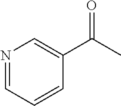 | 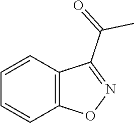 | 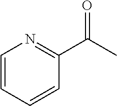 |

TABLE 3
Broad-spectrum teixobactins substituted at positions 3, 4 and 7 of teixobactin.

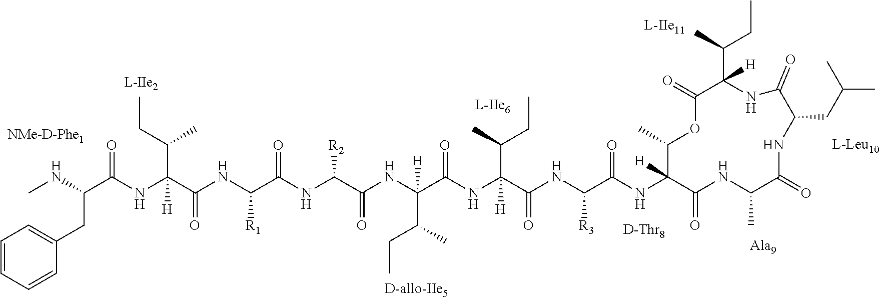

(SEQ ID NO: 6)
R₁, R₂ and/or R₃ Goups

| | | | |
|---|---|---|---|
| 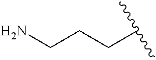 [SMILES: *CCCN] | 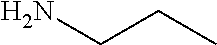 | 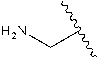 [SMILES: *CN] | 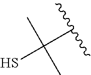 [SMILES: *C(C)(C)S] |
| 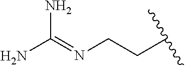 [SMILES: *CCN=C(N)N] | 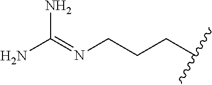 [SMILES: *CCCN=C(N)N] | 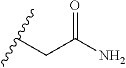 [SMILES: *CC(N)=O] | 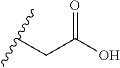 [SMILES: *CC(=O)O] |

These compounds displayed potent antimicrobial activity against teixobactin-resistant Gram-negative clinical isolates while also maintaining their activity against teixobactin-susceptible Gram-positive isolates (Table 4). Polymyxin lipopeptides can act as powerful potentiators for FDA-approved non-antibiotic drugs, by permeabilizing the Gram-negative outer membrane, allowing the non-antibiotic drug to enter the bacterial cell and reach its intracellular targets (Hussein et al. (2018) Comput. Struct. Biotechnol. J. 16:587-599; Hussein et al. (2017) Microbiol. Drug Resist. 23:640-650; Jasim et al. (2017) J. Biomed. Nanotechnol. 13:447-457; Schneider et al. (2016) ACS Infect. Dis. 2:478-488; Tran et al. (2018) Front. Pharmacol. 9:359; Tran et al. (2018) Front. Microbiol. 9:721; Schneider et al. (2017) Essays Biochem. 61:115-125; Diep et al. (2017) Antimicrob. Agents Chemother. 61; Rao et al. (2016) J. Antimicrob. Chemother. 71:3148-3156; Sharma et al. (2017) Int. J. Antimicrob. Agents 49:224-232). Subsequently, this SAR-based design strategy has been expanded to include teixobactins conjugated via labile and non-labile linkers to the polymyxin-lipopeptides (e.g., polymyxin B nonapeptide, FADDI-002 and octapeptin C4) (Velkov et al. (2018) Cell Chem. Biol. 25:380-391 e385; Velkov et al. (2013) Future Microbiol. 8:711-724; Velkov et al. (2014) ACS Chem. Biol. 9:1172-1177; Velkov et al. (2010) J. Med. Chem 53:1898-1916), which generated potent teixobactin lipopeptide hybrids active against teixobactin-resistant PDR Gram-negative ‘superbugs’ (Table 4). Most notably, these novel broad-spectrum teixobactins and their lipopeptide conjugates retained their remarkable resistance-resistant properties against Gram-negative bacteria. In static time-killing studies, UOM-001 (MIC 1 mg/L) at 4×MIC achieved ~6 log 10 kill against a PDR Gram-negative clinical isolate, A. baumannii, with no viable cells detected even at 48 h; no killing was observed with native teixobactin even at 32 mg/L.

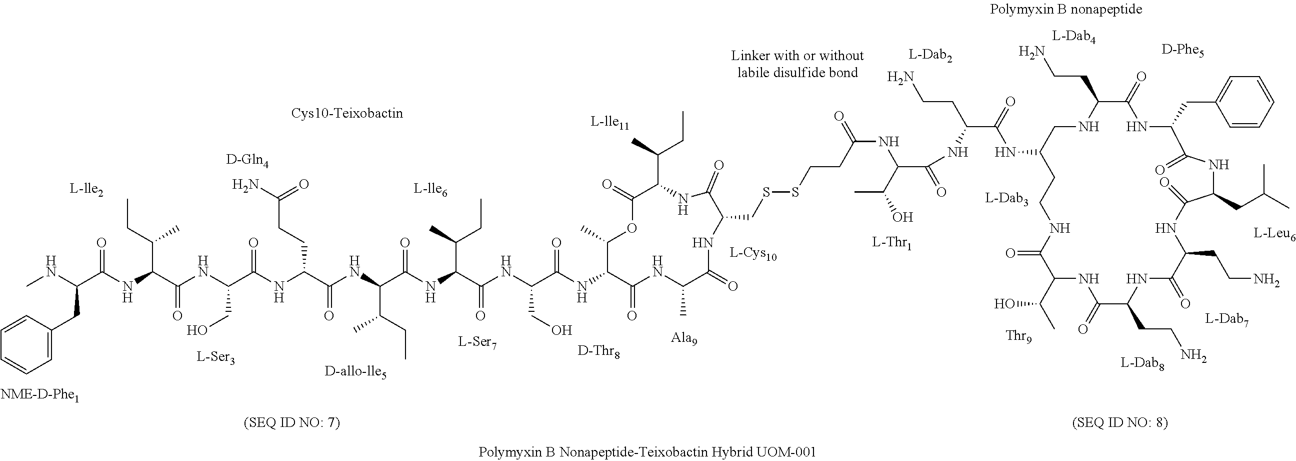

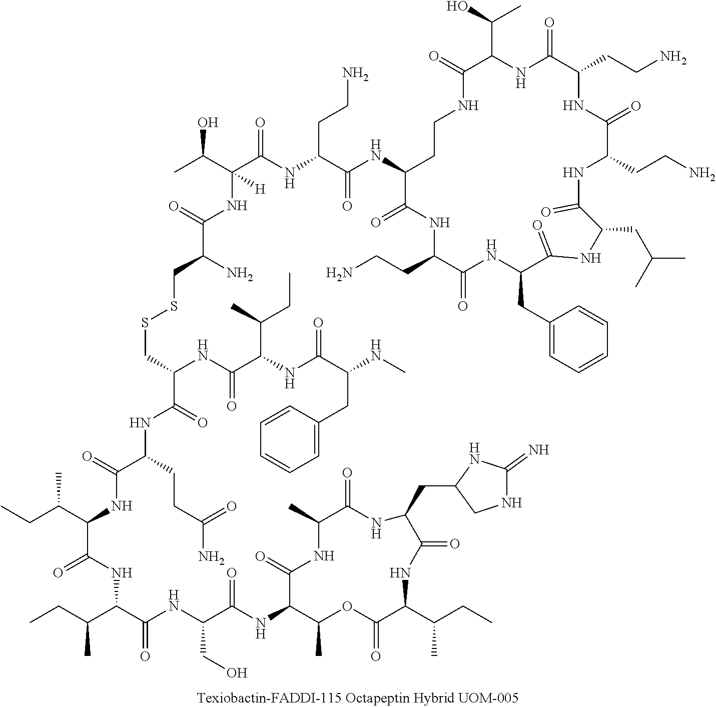 [SMILES: CC[C@H](C)[C@H](NC(=O)[C@@H](Cc1ccccc1)NC)C(=O)N[C@@H](CSSC[C@H](N)C(=O)N[C@H](C(=O)N[C@H](CCN)C(=O)N[C@H]1CCNC(=O)C([C@H](C)O)NC(=O)[C@H](CCN)NC(=O)[C@H](CCN)NC(=O)[C@H](CC(C)C)NC(=O)[C@@H](Cc2ccccc2)NC(=O)[C@@H](CCN)NC1=O)[C@@H](C)O)C(=O)N[C@H](CCC(N)=O)C(=O)N[C@@H](C(=O)N[C@H](C(=O)N[C@@H](CO)C(=O)N[C@H]1C(=O)N[C@@H](C)C(=O)N[C@@H](CC2CNC(=N)N2)C(=O)N[C@@H]([C@@H](C)CC)C(=O)O[C@H]1C)[C@@H](C)CC)[C@@H](C)CC]

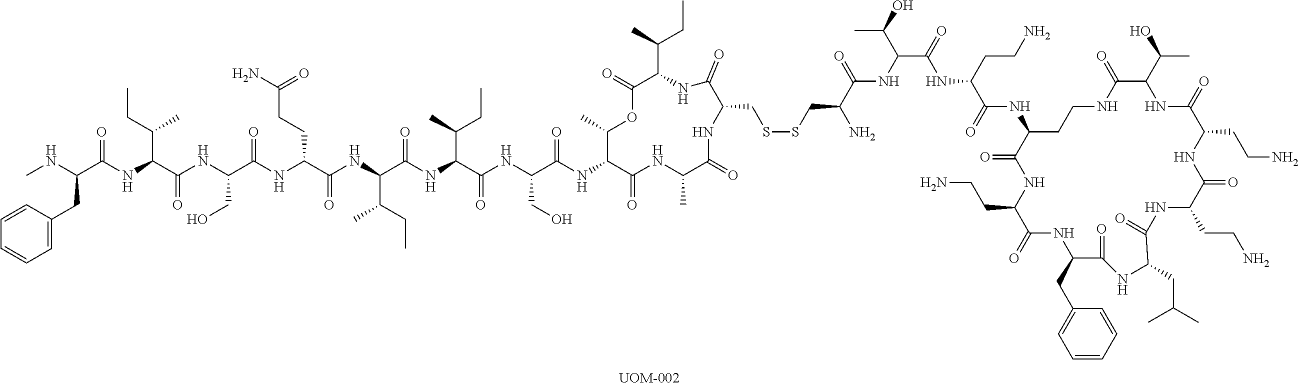

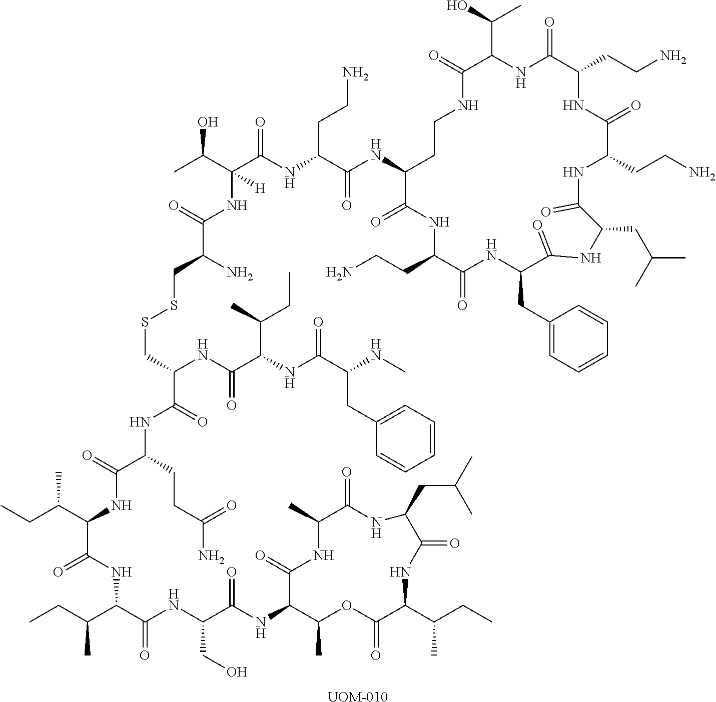

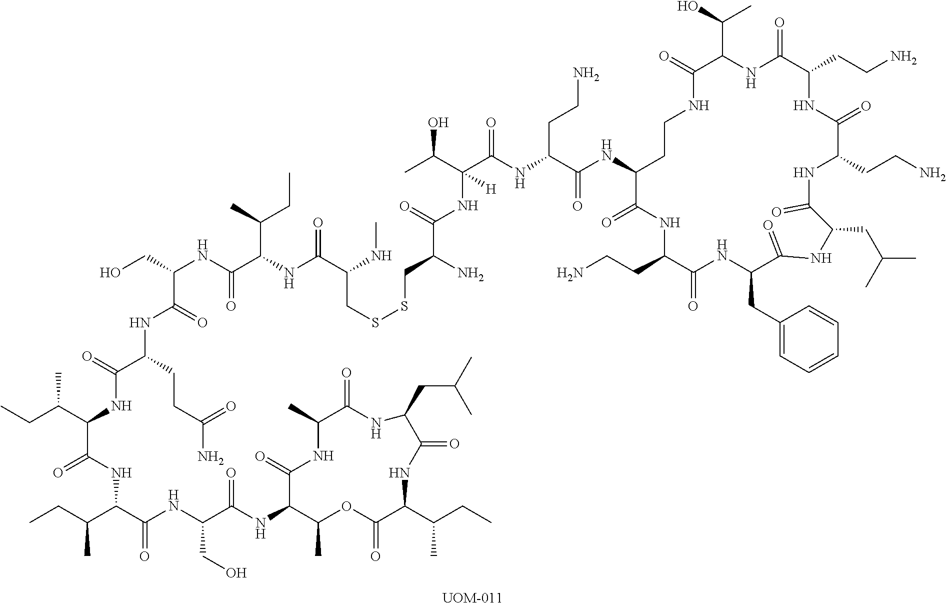

TABLE 4
Strains tested and the MICs of novel broad-spectrum teixobactins
UOM-001, -002, -010, -011, APL-022-024, APL-060-066 and 5 others
novel teixobactin-lipopeptides conjugates compared with colistin.

| | | Total | PDR | | Non-PDR | Teixobactin | |
|---|---|---|---|---|---|---|---|
| | Species | strains | strains | | strains | MICs | |
| Gram- | P. aeruginosa | 16 | 11 | (colistin | 4 & ATCC | 1-4 | mg/L |
| negative | | | 8-128 | mg/L) | 27853 | | |
| | A. baumannii | 7 | 4 | (colistin | 2 & ATCC | 0.5-4 | mg/L |
| | | | 8-128 | mg/L) | 19606 | | |
| | K. pneumoniae | 8 | 5 | (colistin | 2 & ATCC | 0.5-4 | mg/L |
| | | | 32->128 | mg/L) | 13883 | | |
| Gram- | S. aureus | 4 | 3 | (colistin | | 0.5-2 | mg/L |

| | | |
|---|---|---|
| positive | >128 | mg/L) |

Clearly, the SAR-based mechanistic model and lipopeptide hybrid strategy has led to unique opportunities to optimize the teixobactin structure to overcome intrinsic resistance of Gram-negative bacteria to native teixobactin. To date, 88 novel teixobactins and their lipopeptide conjugates have been designed and synthesized using the SAR model and efficient chemistry platform. These compounds have been subjected to preliminary pharmacological evaluations in a proof-of-concept studies as described below.

In vivo Efficacy Study. A proof-of-concept study using a neutropenic mouse lung infection model demonstrated significantly (p<0.045) better in vivo efficacy of the polymyxin B nonapeptide-teixobactin hybrid UOM-010 against a PDR A. baumannii clinical isolate compared with native teixobactin. With an inoculum of 5.23±0.51 log CFU/lung, at 24 h after a single-dose treatment (40 mg/kg), the bacterial burden in the lungs from the mice treated with UOM-010 was 4.21±0.40 log CFU/lung, which was significantly lower than 6.31±0.37 log CFU/lung for native teixobactin and 7.56±0.21 log CFU/lung for the control.

Safety, Tolerability, and in vivo Efficacy. There was no haemolysis in human red blood cells treated with the tested broad spectrum teixobactins or their lipopeptide hybrids at 128 mg/L (the highest concentration examined). After administration of UOM-001, -002, -010, and -011 to rats (intravenous, 0.75 mg/kg) and mice (subcutaneous, 40 mg/kg), no adverse effects were observed. A preliminary in vitro study was performed to examine the effect on human keratinocytes and murine fibroblast cells. Similar to native teixobactin, UOM-001, -002, -010, and -011 at 1.5, 5, 15 and 50 mg/L had little effect over 48 h on the morphology of fibroblasts (3T3) and keratinocytes (HaCaT).

It is believed this is the first application of an SAR-based mechanistic model to discover novel teixobactins against Gram-negative ‘superbugs’ which are intrinsically resistant to native teixobactin.

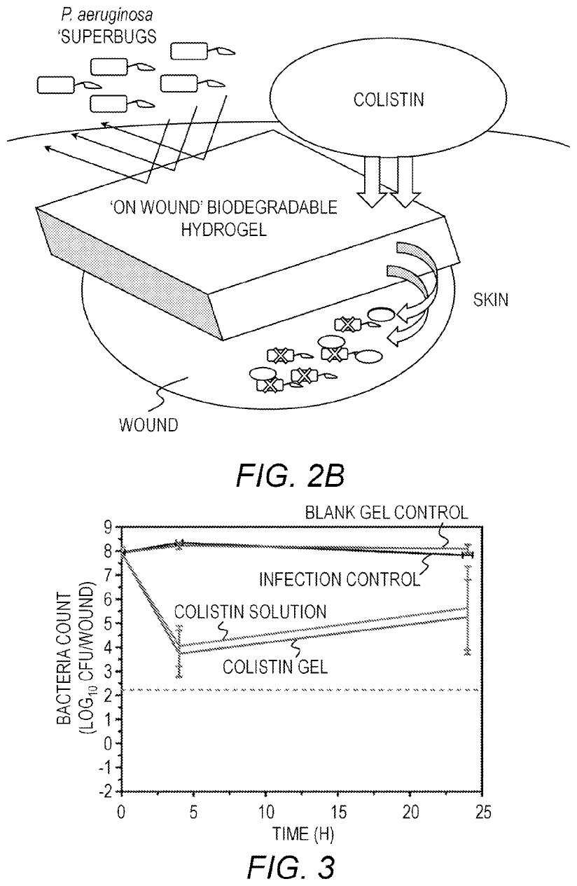

Synthesis of a Chitosan and γ-Cyclodextrin Antibiotic Hydrogels and Testing in a Mouse Burn Infection Model. Modern dressings have been designed to provide an environment around the wound that is most conducive to wound healing (Elsner, et al. (2010) J Biomed Mater Res B Appl Biomater 93:425-435). These dressings provide a controlled moist environment, control exudates and limit exposure of the wound to further infection. The advantages of such a system include combining the drug delivery with the protection of the wound and ability to achieve sustained delivery. An important issue to address with this system includes stability of the active ingredient and its diffusion from the dressing into the tissue. To this end, a chitosan-colistin hydrogel was synthesized using glycol chitosan, PEG4000-DF and colistin (FIG. 2A); and an animal burn infection model was used to evaluate the gel performance in vivo (FIG. 2B).

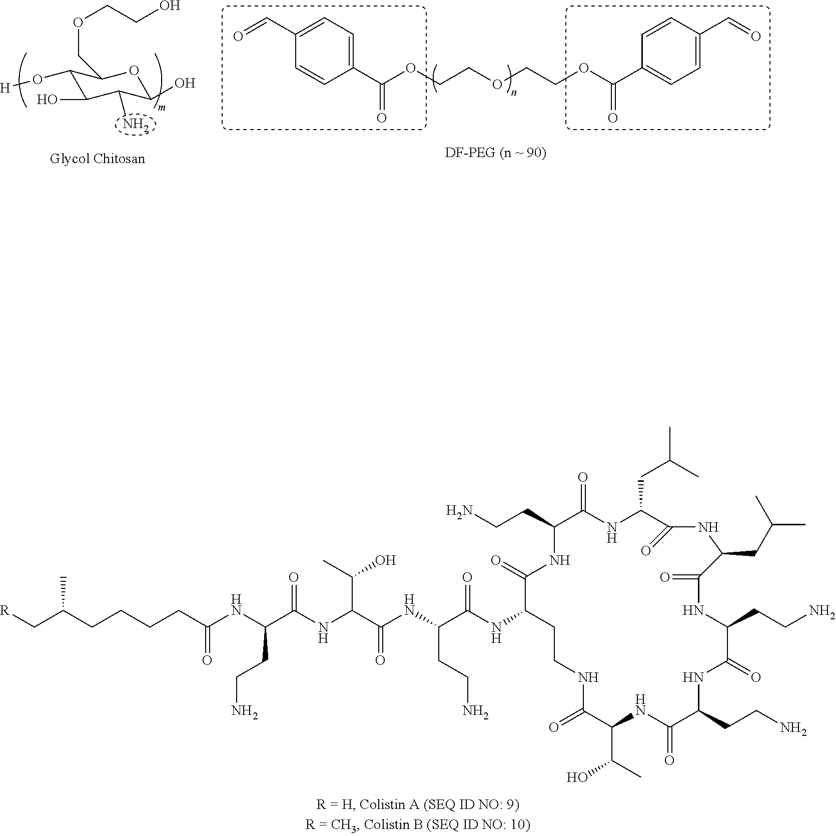

The chitosan-colistin hydrogel is an inexpensive, self-healable and highly biocompatible material which provides up to 95% colistin release within 24 hours and showed excellent in vitro activity against P. aeruginosa in a disc diffusion assay and mouse burn infection model (FIG. 3). The physical properties of the hydrogel were unaffected by colistin, this allowed loading of a wide range of colistin concentrations into the hydrogel matrix without impacting its size. Serendipitously, the hydrogel formation process was accelerated in the presence of colistin. The chitosan-colistin hydrogel dressing (containing 0.3 mg colistin) displayed excellent in vivo activity, producing a ~4-log reduction in the bacterial load in a burn wound (1 cm²) infection, established in mice by inoculating 100 μL 10⁸ CFU/mL of P. aeruginosa ATCC 27853 (FIG. 3). A γ-cyclodextrin-polymyxin B hydrogel formulation was also developed which displayed excellent in vitro activity in static time-kill studies against P. aeruginosa and in vivo in a mouse infection model. These hydrogel formulations have the advantage in that the complex is non-covalent in nature and highly stable at room temperature.

Their development as a hydrogel wound dressing will represent a first-in-class broad-spectrum resistance-proof, battle-field ready, antibiotic treatment for PDR combat wound infections. Therefore, the invention will have a significant impact on treatment of bacterial infections worldwide, especially war-zone related wound infections.

TABLE 5
Chemical modifications and rationale for each teixobactin series.

| | Chemical | |
|---|---|---|
| Series | Modifications | Rationale |
| 1 | Lipidic | Modifications: (1) Combinations of C6 to C8 fatty acyl, |
| | (N-terminus: | heteroaryl, halogenated, and lipidic aromatic groups. (2) |
| | Positions 1, 2, 3) | Various alternate alkyl (branched, unsaturated) and/or |
| | | aromatic moieties. (3) Lipidic amino acids. |
| | | Rationale: To provide substantially stronger hydrophobic |
| | | interactions with the prenyl chain of lipid II and enhance |
| | | Gram-negative outer membrane anchoring/penetration. |
| 2 | Mixed Lipidic | Modifications: Combinations of lipidic modification above |
| | and Polar | with: (1) heteroaryl, halogenated aromatic groups (2) one or |
| | (N-terminus: | more H-bonding residues (Thr, Ser), (3) alternate basic |
| | Positions 1, 2, 3) | residues (Lys, Orn, Arg, His), acidic residues (Glu, Asp), or |
| | | (4) H-bonding residues (Asn, Gln). |
| | | Rationale: To optimize the Gram-negative outer membrane |
| | | anchoring/penetration; which is known to require polar |
| | | interactions with lipid A and other outer membrane |
| | | phospholipidsª. |
| 3 | Lipopeptide | Modifications: The superior teixobactins identified in Series |
| | coupling | 1 and 2 are coupled to the lipopeptide potentiators using labile |
| | | and non-labile linkers: polymyxin B nonapeptide, |
| | | FADDI-002^{b} or octapeptins C4^{c}. |
| | | Rationale: To enhance Gram-negative outer membrane |
| | | permeabilising activity thereby allowing higher concentrations |
| | | of the teixobactin to reach its intracellular lipid II target. |
| 4 | Ring Modification | Modification: Teixobactin, a native antibiotic, incorporates |
| | (C-terminus: | non-natural amino acids and engages with lipid II, thereby |
| | Position 8, 9, | facilitating the synthesis of mimetic compounds. |
| | 10, 11) | Rationale: The facile synthesis of teixobactin and its |
| | | augmentation of Gram-negative outer membrane |
| | | permeabilization activity serve to facilitate increased |
| | | concentrations of the antibiotic reaching its intracellular |
| | | lipid II target. |
| 5 | Lipidic | Modification: Introducing positive charge replacing Ser3, |
| | (N-terminus: | Gln4 and Ser7 by Lys, Orn, Dab, Dap. |
| | Positions 3, 4, 7) | Rationale: To augment the permeability of cellular |
| | | membranes and improve solubility. |

^{a}Nikaido (2003) MMBR 67:593-656. ^{b}Velkov et al. (2014) ACS Chem. Biol. 9:1172-1177. ^{c}Velkov et al. (2018) Cell Chem. Biol. 25(4):380-391.

### Example 2. Dry Powder Formulations

The present example shows that teixobactin-lipopeptide hybrids delivered as a dry powder inhalation have significantly improved efficacy for the treatment of lung infections by virtue of their unique mode of action, no detectable resistance, high local exposure in the lungs with low systemic exposure and low toxicity. Importantly, the hybrids displayed superior in vivo efficacy compared to treatment with the combination of the individual compounds or each compound per se. This is a significant development in the field as the teixobactin-lipopeptide hybrid represents the first-in class broad-spectrum ‘resistance-proof’ dry powder inhalation antibiotic for the treatment of MDR bacterial lung infections.

Colistin and polymyxin B are increasingly used in the United States and many other countries. Worryingly, a clinical pharmacokinetics/pharmacodynamic/toxicodynamic (PK/PD/TD) study on colistin in 230 critically-ill patients showed that the efficacy of parenteral colistin is disappointing for treatment of pulmonary infections due to very limited exposure at the infection site. Simply increasing the dose of parenteral polymyxins is not a viable option because of dose-limiting nephrotoxicity; and even worse, suboptimal use of parenteral polymyxins can lead to resistance. Similarly, the presented studies in rats revealed that intravenous teixobactin exhibited very low drug exposure in the lungs.

Applicant has investigated polymyxin SAR/STR, as well as the interconnection of polymyxin lipopeptides. SAR- and STR-based mechanistic models were also generated which allowed for rational design of novel, safer polymyxin-like lipopeptides with superior activity against Gram-negative bacteria compared to polymyxin B and colistin. The development of these lipopeptides in polymyxin discovery projects has also been completed. These superior lipopeptides are coupled to teixobactins to generate safe, resistance-proof broad-spectrum hybrids. The first SAR model for teixobactins was also developed (FIG. 4). The breakthrough understanding of SAR/STR allows performing of careful tailoring of the teixobactin and polymyxin lipopeptide scaffolds for modulating pharmacological properties and toxicity without compromising their antibacterial activity. Using this rational SAR/STR-driven design approach, it is believed this is the first development of novel, safe teixobactin-lipopeptide hybrids UOM-001 (teixobactin-polymyxin B hybrid) and -005 (texiobactin-FADDI-115 octapeptin hybrid) with broad-spectrum antibacterial activity against Gram-positive and Gram-negative ‘superbugs’.

An Integrated Teixobactin-Lipopeptide Hybrid Antibiotic Discovery and Development Platform. As described herein, a highly efficient antimicrobial peptide discovery and development platform was established that integrates medicinal chemistry (SAR/STR models, total synthesis), microbiology (a large selection of PDR clinical isolates) and pharmacology (multi-omics, rodent models for toxicity, PK, lung infection) to rapidly undertake lead optimization and select candidates for advanced pre-clinical evaluations. Efficient in vivo models for screening compounds have been developed along with very efficient rodent lung toxicity models, which rapidly presents with reproducible lung toxicity (two days) using a minimal amount of compound. DPI formulation of antibiotics such as polymyxins have been developed. Notably, employing modern particle engineering techniques (e.g., particle surface coating) have been used to develop powder aerosol formulations of colistin and its combinations with other antibiotics. Successfully employed innovative dry particle coatings to improve the aerosol efficiency and manufacturability of powder aerosol formulations for antibiotics. These particle engineering techniques and advanced characterization technologies are employed to develop novel formulations of the hybrids which possess superior delivery performance and improved stability characteristics.

Octapeptins are naturally occurring lipopeptides that structurally resemble polymyxins, and uniquely display good antibacterial activity against polymyxin-resistant Gram-negative bacteria. The first SAR model for the octapeptins was constructed, which rationalizes how they overcome polymyxin resistance (FIG. 4). This model has been employed to develop the first safe and effective octapeptins (e.g., FADDI-115) that target polymyxin resistance. These novel octapeptins are coupled to teixobactin to develop hybrid molecules that are highly active against polymyxin-resistant PDR Gram-negatives (Table 5, Compound Series 3).

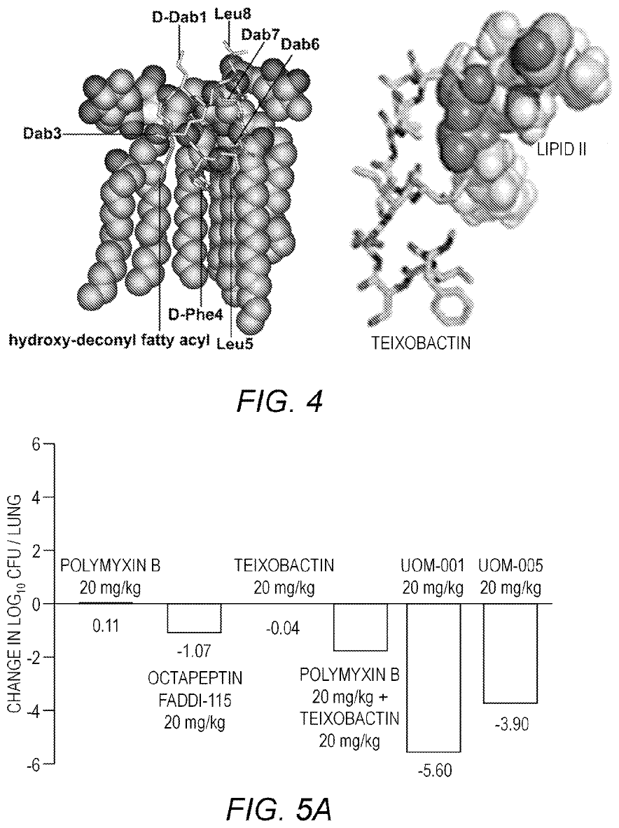

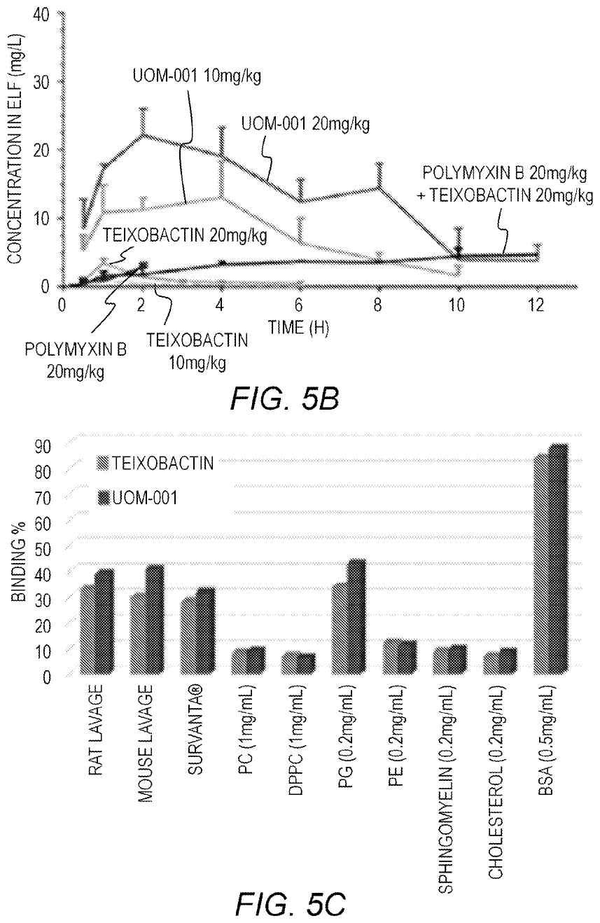

Inhaled Teixobactin-Lipopeptide Hybrids for Lung Infections. Pulmonary infections caused by PDR K. pneumoniae, P. aeruginosa, A. baumannii, S. aureus and S. pneumoniae are particularly difficult to treat. The aim is to develop an inhaled teixobactin-lipopeptide hybrid, which offers significantly improved efficacy for the treatment of Gram-negative and Gram-positive PDR lung infections by virtue of the high local exposure in the lungs, low propensity for resistance, systemic exposure and low toxicity; which together translate into a significant advantage over native teixobactin or lipopeptides. Findings highlighted that the UOM-001 and -005 hybrid DPI displayed superior in vivo efficacy and achieved much higher epithelial lining fluid (ELF) exposure in the lungs compared to the individual teixobactin and lipopeptide components (FIG. 5A-5C). Notably, both teixobactin and the UOM-001 hybrid exhibited superior lung exposure when delivered directly as a dry powder; whereas parenteral delivery resulted in very low (i.e., sub-MIC) levels in the lungs. The hybrids offer an additional advantage over native teixobactin per se as it combines the latter with a lipopeptide with a different MOA of bacterial killing. This makes it markedly more difficult for the bacteria to develop resistance as the bacterial cell is challenged with two antibiotics with different killing mechanisms.

Optimized Chemical Synthesis Of Teixobactin-Lipopeptide Hybrids. The primary interest in teixobactin relates to broadening its spectrum of activity through the hybridization technology, whilst preserving its resistance-proof properties. This necessitates ready access to analogues to conduct a thorough design principle based on the SAR models and lipopeptide coupling strategies. It is therefore imperative that the teixobactin synthetic route is rapid and efficient. To this end, a convergent synthetic strategy was devised, which can achieve preparation of large quantities of both the N-terminal linear protected fragment and the C-terminal macrocycle. It would thus only be necessary to synthesize modifications of these shorter precursors to generate new teixobactin analogs (versus performing a full stepwise assembly), expediting preparation of the teixobactin-lipopeptide candidate UOM-001 in >100 mg quantities for formulation and disposition studies. The cyclization/deprotection yield of the purified linear teixobactin peptide is 75%, which is believed to be the highest reported and highlights the efficiency of this strategy. The rapid synthetic protocol provides ready access to teixobactin analogues with high purity (>95%).

Discovery of Novel and Safe Broad-Spectrum Teixobactin-Lipopeptide Hybrids. A comprehensive teixobactin SAR review to date was published, which provides a complete compendium of the hundreds of teixobactin analogues that have been reported in the scientific literature and patents. Notably, all teixobactin discovery programs have been empirical in nature and none were specifically driven by a SAR approach nor was significant activity against Gram-negative pathogens targeted. Another major knowledge-gap limiting the progression of the field is the lack of understanding of structure-conformation relationships of teixobactin interactions with its lipid II target. This experimentally based teixobactin SAR model developed (FIG. 4), that incorporates all prior teixobactin SAR for the rational design of novel broader-spectrum teixobactins, has allowed development of novel teixobactin SAR which to date have not been reported in the literature. Importantly, this novel teixobactin-lipopeptide hybrid design strategy (Table 5) is driven by the intelligent SAR model, which incorporates feed-back loops to ensure subsequent compound iterations develop improvements in efficacy and safety. This innovative rational-design approach ensures success, unlike current empirical programs, which have yet to yield compounds with improved activity or an expanded antibacterial spectrum over the natural product. Polymyxin lipopeptides can act as powerful potentiators for FDA-approved non-antibiotic drugs, by permeabilizing the Gram-negative outer membrane allowing the non-antibiotic drug to enter the bacterial cell and reach its intracellular target(s). Intrinsic impermeability of the Gram-negative outer membrane towards teixobactin has been overcome by coupling polymyxin-like lipopeptides (e.g., FADDI-115 and octapeptin C4) onto the teixobactin molecule via a labile linker to generate broad-spectrum hybrids highly active against both PDR Gram-positives and teixobactin-resistant PDR Gram-negative ‘superbugs’ (Table 5). Notably, these lipopeptides (e.g., FADDI-115 and octapeptin C4), unlike polymyxins, are broad-spectrum and possess potent activity against Gram-positives as well as Gram-negatives. The MOA studies against Gram-negatives indicate that the lipopeptide permeabilizes and/or disrupts the outer membrane, allowing teixobactin to enter the cell and reach its intracellular lipid II/III target; whereas against Gram-positives there is no permeability barrier for either the teixobactin or the lipopeptide, which are free to act against their respective targets. So it follows, irrespective of whether the target bacteria are Gram-negative or Gram-positive, the combined and robust MOA of the hybrid prevents the emergence of resistance. In static time-kill studies, UOM-001 and -005 at 1×MIC (MIC 1 mg/L) achieved ~6 log 10 kill against a polymyxin-resistant P. aeruginosa isolate, FADDI-PA021m from the lung of a cystic fibrosis patient. Notably, treatment with UOM-001 or -005 achieved complete bacterial killing and no visible bacterial regrowth was detected even at 48 h; whereas no killing was observed with native teixobactin or polymyxin B (MICs>128 mg/L). Serial passaging of FADDI-PA021m in media spiked with 0.5×MIC UOM-001 or -005 for 21 days failed to produce resistant mutants. These SAR-based mechanistic models have led to unique opportunities to design teixobactin-lipopeptide hybrids with superior antibacterial activity compared to the individual teixobactin or lipopeptide components.

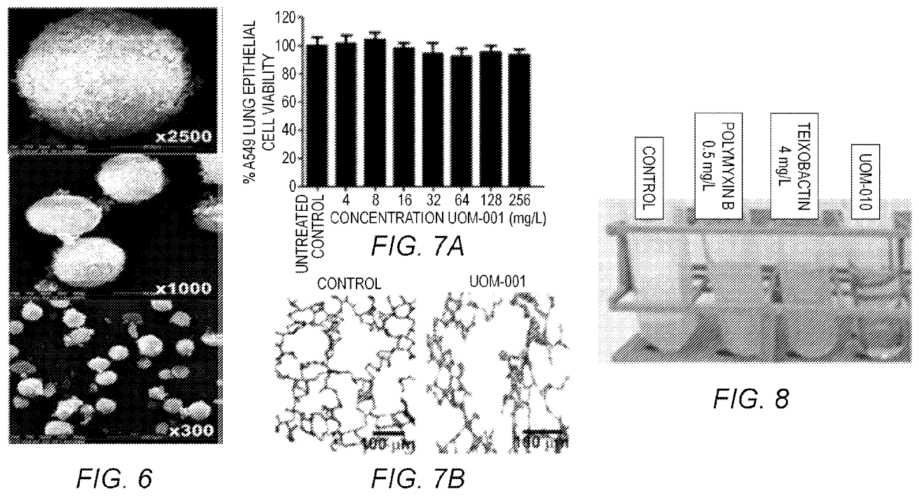

Dry Powder Formulation and in vivo Efficacy of UOM-001 and UOM-005 in a Lung Infection Model. Novel DPI formulations have been developed for native teixobactin, UOM-001 and -005 with good stability and in vitro aerosol performance (inhalable fine particle fraction [FPF]>70) (FIG. 6). Notwithstanding, scanning electron microscopy (SEM) imaging of the UOM-001 DPI indicated some particle aggregation occurs. The activity of DPI UOM-001 and comparator antibiotics was assessed against a challenge panel consisting of 207 P. aeruginosa isolates collected from different parts of the world; 105 isolates were enriched for resistance against β-lactam/β-lactamase inhibitor combinations (Table 6). UOM-001 (MIC50/90, 0.25/1 mg/L) was the most potent antibiotic and was 4-fold more potent than colistin (MIC50 1 mg/L). Importantly, in vivo studies have been conducted that conclusively demonstrate the hybrids UOM-001 (teixobactin-polymyxin B hybrid) and -005 (texiobactin-FADDI-115 octapeptin hybrid) display superior activity compared to treatment with the combination of the individual compounds or each compound per se (FIG. 5A-5C). In the neutropenic rat lung infection model, DPI UOM-001 and -005 (MICs 1 mg/L) demonstrated significantly (p<0.045) better in vivo efficacy at a dose of 20 mg/kg delivered directly to the lungs using an insufflator device PenWu (Bio Jane, Shanghai, China) against a polymyxin-resistant cystic fibrosis isolate P. aeruginosa FADDI-PA021m; compared to the same DPI dose of the combination of teixobactin+polymyxin B, teixobactin+octapeptin FADDI-115 or the individual antibiotics. The teixobactin-lipopeptide hybrids have a labile linker between the two antibiotic components that allows for sustained release of each component at the infection site simultaneously, thereby targeting their synergistic activity. Whereas the individual components when administered together likely display different disposition, uptake, and elimination characteristics, and therefore become diffusely distributed from each other, resulting in the loss of antibacterial synergy. In the rat PK study, direct lung delivery of the DPIs (20 mg/kg) using a Pen Wu insufflator device showed that the epithelial lining fluid (ELF) exposures for the UOM-001 hybrid were significantly greater compared to the individual components or their combination (FIG. 5B). While in plasma, a low Cmax (UOM-001 2.0 μg/mL; teixobactin 2.5 μg/mL; lipopeptide 2.0 μg/mL) was achieved, indicating that pulmonary delivery resulted in desired localization in the lungs with low systemic exposure. In contrast, following intravenous administration of an identical dose, the ELF concentration of UOM-001 was below the limit of quantification (0.1 μg/mL). These findings clearly demonstrate that the UOM-001 hybrid DPI displayed superior in vivo efficacy and achieved much higher ELF exposure in the lungs compared to the individual teixobactin and lipopeptide components. Pulmonary surfactant is a primary component of the ELF which can bind antibiotics that can impede the antibacterial activity of the drug. The binding of UOM-001 and teixobactin (5 mg/L for 4 h 37° C.) to lung surfactant or its individual components was determined by ultracentrifugation and LC-MS (FIG. 5C). The low binding of UOM-001 to lung surfactant is consistent with its good efficacy in the lung infection model. The effect of surfactant on the in vitro antibacterial activity of UOM-001 was assessed with the use of three polymyxin-resistant P. aeruginosa cystic fibrosis isolates. Beractant (Survanta™; Abbott Laboratories) served as the pulmonary surfactant. Beractant is a bovine-derived surfactant containing 25 mg/mL phospholipids. Although its composition differs slightly from that of human surfactant, it is functionally equivalent. For surfactant testing, the bacterial growth medium was supplemented with 10% beractant (=2.5 mg/mL). Since the surfactant produces turbidity, the minimum bactericidal concentration (MBC) was used to compare potency of UOM-001 in the absence or presence of surfactant. The MBCs of UOM-001 (0.125-0.5 mg/L) were not affected even when tested with the highest concentrations of surfactant (2.5 mg/mL, 10%). Importantly, UOM-001 also displayed potent anti-biofilm effect and bacterial killing against a heavy biofilm forming cystic fibrosis isolate P. aeruginosa FADDI-PA021m (MIC 1 mg/L), even with growth in artificial sputum media. Overall, the data demonstrate that the DPIs of UOM-001 and -005 have excellent activity in a lung infection model, excellent aerosol performance, stability, low lung surfactant binding and excellent penetration/exposure in the airways, compared to intravenous administration.

TABLE 6
Activity of UOM-001 and comparators (as
MIC₅₀/MIC₉₀) vs. PDR P. aeruginosa (n = 207)

| | | CAZ- | | | | PIP- | | UOM- |
|---|---|---|---|---|---|---|---|---|
| | MER | AVI | AMK | GNT | LEVO | TAZ | COL | 001 |
| MIC₅₀ | 16 | 2 | 16 | 8 | 8 | 64 | 1 | 0.25 |
| MIC₉₀ | >32 | 8 | >32 | >16 | >16 | >128 | 1 | 1 |

MER, meropenem; CAZ- AVI, ceftazidime-avibactam; AMK, amikacin; GNT, gentamicin; LEVO, levofloxacin; PIP-TAZ, piperacillin-tazobactam; COL, colistin.

Safety and Tolerability. There was no haemolysis in human red blood cells treated with any of the teixobactin-lipopeptide hybrids at 128 mg/L (the highest concentration examined). Moreover, UOM-001 and -005 showed excellent stability in human plasma (no degradation over 24 h, no genotoxicity, AMES test) and no hERG receptor activity. The maximum tolerated dose (MTD) of UOM-001 and -005 following intravenous administration to rats was 50 mg/kg and 40 mg/kg (n=6), respectively, indicating the hybrids are very safe systemically within the therapeutic range and free from acute adverse effects. An in vitro study was performed to investigate any potential toxicity on human A549 lung epithelial cells (FIG. 7A); UOM-001 up to 256 mg/L had no effect over 48 h on the viability of the A549 cells. In rats, DPI UOM-001 did not display any histological lung damage (semi-quantitative score [SQS] of zero) following a single dose (20 mg/kg, n=4) (FIG. 7B).

Discussion Detectable resistance to native teixobactin in MRSA has not been identified despite serial passage to sub-inhibitory doses for 27 days. Similarly, no resistance was detected against the UOM-001 hybrid in a polymyxin resistant P. aeruginosa FADDI-PA021m isolate from the lung of a cystic fibrosis patient following growth in media spiked with 0.5×MIC UOM-001 for 21 days (FIG. 8). To date there has been only one report of S aureus resistance against a non-native Arg10-teixobactin analog following serial passaging for 75 days. Notably, the nature of the resistance was linear, extremely slow and mediocre at best with only a 2.4-fold increase in MIC. The magnitude of evolved resistance to Arg10-teixobactin was 300-fold and 2,500-fold less than the moenomycin A and rifampicin controls, respectively. Clearly, the development of resistance, if any, against teixobactins is extremely costly and was rapidly lost in the absence of antibiotic selection. Similarly, resistance to polymyxin B and colistin can cause significant biological fitness cost in bacteria. The hybrid molecules offer the advantage of incorporating polymyxin-like lipopeptides which are highly active against polymyxin-resistant strains, which means that once again resistance to the hybrid cannot be easily developed as it would be even more costly for the bacteria in terms of biological fitness. In summary, the discovery of novel, potent, broad-spectrum teixobactin-lipopeptide hybrids holds immense potential for the development of therapeutics against PDR bacteria and addresses the impending threat of a post-antibiotic world. Delivering a teixobactin-polymyxin lipopeptide hybrid directly to the site of infection in the airway as a DPI formulation is a highly effective approach for treating lung infections. The data from the experiment herein revealed that the inhaled teixobactin-polymyxin lipopeptide hybrid UOM-001 achieved superior local drug exposure and enhanced bacterial killing with negligible systemic toxicity in a rat lung infection model. (FIG. 5A-5C).

### Example 3. Teixobactin Analogues

Additional teixobactin analogues were prepared with modifications at the C-terminus and Positions 3, 4, and 7 (Table 7). These teixobactin analogues can be used in the hybrid molecules described herein.

TABLE 7
Teixobactin analogues.

| | SEQ |
|---|---|
| | ID |
| Analogue | NO: |
| 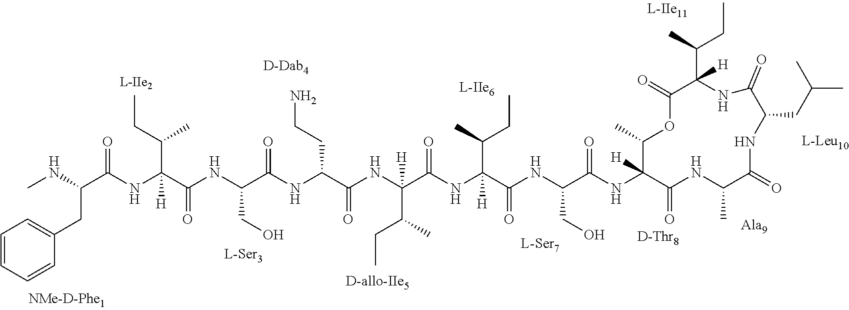 [SMILES: CC[C@@H](C)[C@@H](NC(=O)[C@@H](CCN)NC(=O)[C@H](CO)NC(=O)[C@@H](NC(=O)[C@H](Cc1ccccc1)NC)[C@@H](C)CC)C(=O)N[C@H](C(=O)N[C@@H](CO)C(=O)N[C@H]1C(=O)N[C@@H](C)C(=O)N[C@@H](CC(C)C)C(=O)N[C@@H]([C@@H](C)CC)C(=O)O[C@H]1C)[C@@H](C)CC] | 11 |
| APL-021 | |
| 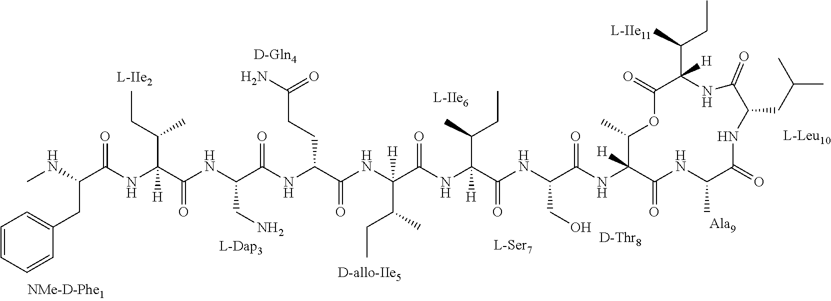 [SMILES: CC[C@@H](C)[C@@H](NC(=O)[C@@H](CCC(N)=O)NC(=O)[C@H](CN)NC(=O)[C@@H](NC(=O)[C@H](Cc1ccccc1)NC)[C@@H](C)CC)C(=O)N[C@H](C(=O)N[C@@H](CO)C(=O)N[C@H]1C(=O)N[C@@H](C)C(=O)N[C@@H](CC(C)C)C(=O)N[C@@H]([C@@H](C)CC)C(=O)O[C@H]1C)[C@@H](C)CC] | 12 |
| APL-022 | |
| 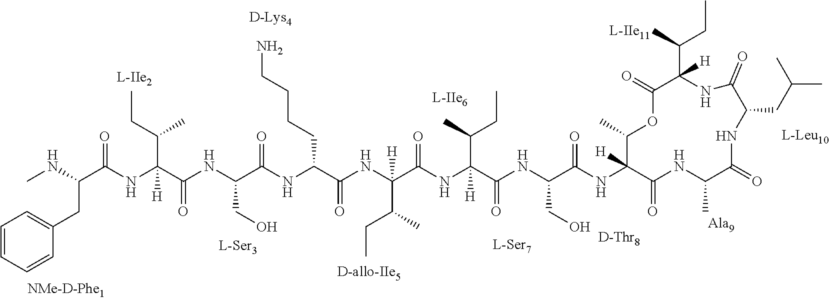 [SMILES: CC[C@@H](C)[C@@H](NC(=O)[C@@H](CCCCN)NC(=O)[C@H](CO)NC(=O)[C@@H](NC(=O)[C@H](Cc1ccccc1)NC)[C@@H](C)CC)C(=O)N[C@H](C(=O)N[C@@H](CO)C(=O)N[C@H]1C(=O)N[C@@H](C)C(=O)N[C@@H](CC(C)C)C(=O)N[C@@H]([C@@H](C)CC)C(=O)O[C@H]1C)[C@@H](C)CC] | 13 |
| APL-023 | |
| 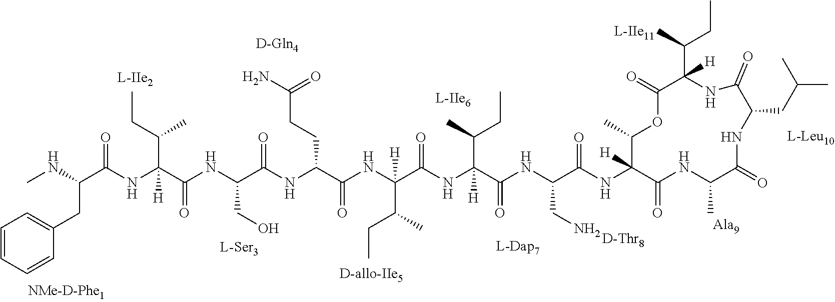 [SMILES: CC[C@@H](C)[C@@H](NC(=O)[C@@H](CCC(N)=O)NC(=O)[C@H](CO)NC(=O)[C@@H](NC(=O)[C@H](Cc1ccccc1)NC)[C@@H](C)CC)C(=O)N[C@H](C(=O)N[C@@H](CN)C(=O)N[C@H]1C(=O)N[C@@H](C)C(=O)N[C@@H](CC(C)C)C(=O)N[C@@H]([C@@H](C)CC)C(=O)O[C@H]1C)[C@@H](C)CC] | 14 |
| APL-024 | |
| 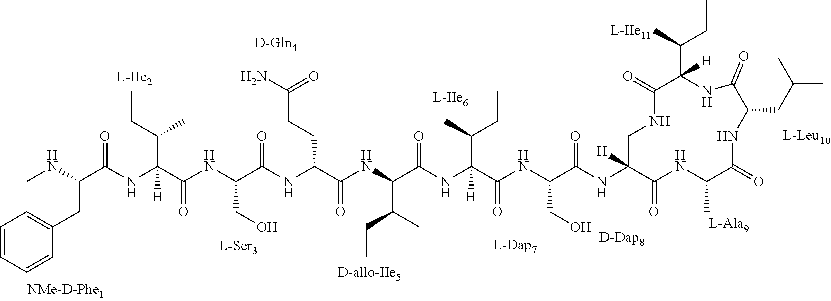 [SMILES: CC[C@H](C)[C@@H]1NC(=O)[C@H](CC(C)C)NC(=O)[C@H](C)NC(=O)[C@H](NC(=O)[C@H](CO)NC(=O)[C@@H](NC(=O)[C@H](NC(=O)[C@@H](CCC(N)=O)NC(=O)[C@H](CO)NC(=O)[C@@H](NC(=O)[C@H](Cc2ccccc2)NC)[C@@H](C)CC)[C@@H](C)CC)[C@@H](C)CC)CNC1=O] | 15 |
| APL-041 | |
| 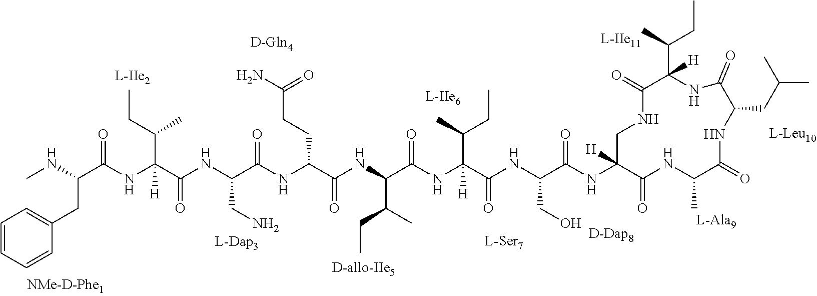 [SMILES: CC[C@H](C)[C@@H]1NC(=O)[C@H](CC(C)C)NC(=O)[C@H](C)NC(=O)[C@H](NC(=O)[C@H](CO)NC(=O)[C@@H](NC(=O)[C@H](NC(=O)[C@@H](CCC(N)=O)NC(=O)[C@H](CN)NC(=O)[C@@H](NC(=O)[C@H](Cc2ccccc2)NC)[C@@H](C)CC)[C@@H](C)CC)[C@@H](C)CC)CNC1=O] | 16 |
| APL-042 | |
| 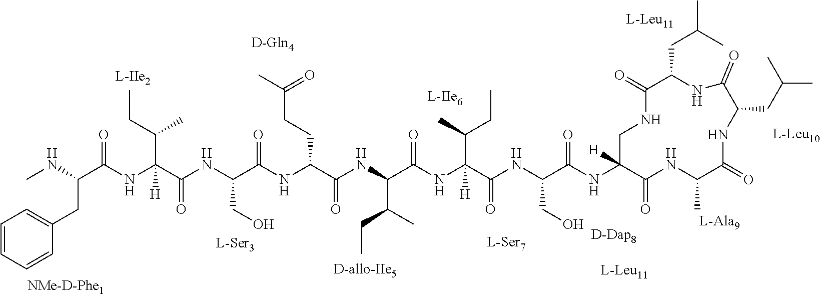 [SMILES: CC[C@H](C)[C@H](NC(=O)[C@H](Cc1ccccc1)NC)C(=O)N[C@@H](CO)C(=O)N[C@H](CCC(C)=O)C(=O)N[C@@H](C(=O)N[C@H](C(=O)N[C@@H](CO)C(=O)N[C@@H]1CNC(=O)[C@H](CC(C)C)NC(=O)[C@H](CC(C)C)NC(=O)[C@H](C)NC1=O)[C@@H](C)CC)[C@@H](C)CC] | 17 |
| APL-047 | |
| 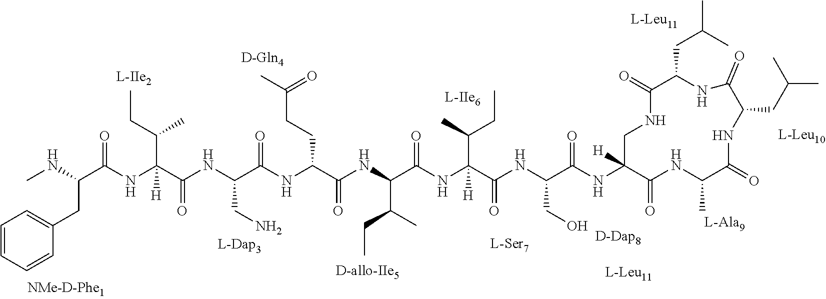 [SMILES: CC[C@H](C)[C@H](NC(=O)[C@H](Cc1ccccc1)NC)C(=O)N[C@@H](CN)C(=O)N[C@H](CCC(C)=O)C(=O)N[C@@H](C(=O)N[C@H](C(=O)N[C@@H](CO)C(=O)N[C@@H]1CNC(=O)[C@H](CC(C)C)NC(=O)[C@H](CC(C)C)NC(=O)[C@H](C)NC1=O)[C@@H](C)CC)[C@@H](C)CC] | 18 |
| APL-048 | |
| 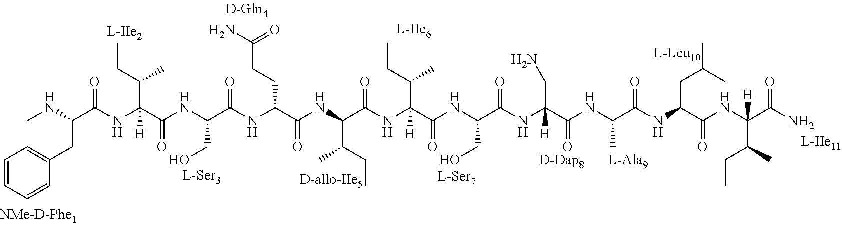 [SMILES: CC[C@H](C)[C@H](NC(=O)[C@H](CC(C)C)NC(=O)[C@H](C)NC(=O)[C@@H](CN)NC(=O)[C@H](CO)NC(=O)[C@@H](NC(=O)[C@H](NC(=O)[C@@H](CCC(N)=O)NC(=O)[C@H](CO)NC(=O)[C@@H](NC(=O)[C@H](Cc1ccccc1)NC)[C@@H](C)CC)[C@@H](C)CC)[C@@H](C)CC)C(N)=O] | 19 |
| APL-060 | |
| 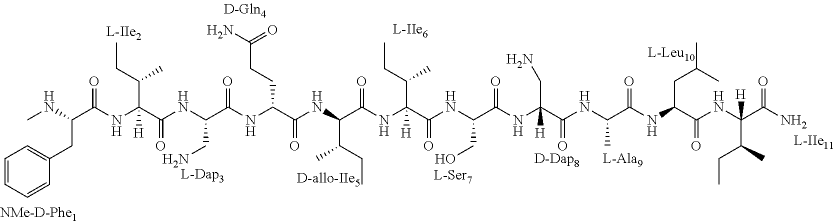 [SMILES: CC[C@H](C)[C@H](NC(=O)[C@H](CC(C)C)NC(=O)[C@H](C)NC(=O)[C@@H](CN)NC(=O)[C@H](CO)NC(=O)[C@@H](NC(=O)[C@H](NC(=O)[C@@H](CCC(N)=O)NC(=O)[C@H](CN)NC(=O)[C@@H](NC(=O)[C@H](Cc1ccccc1)NC)[C@@H](C)CC)[C@@H](C)CC)[C@@H](C)CC)C(N)=O] | 20 |
| APL-061 | |
| 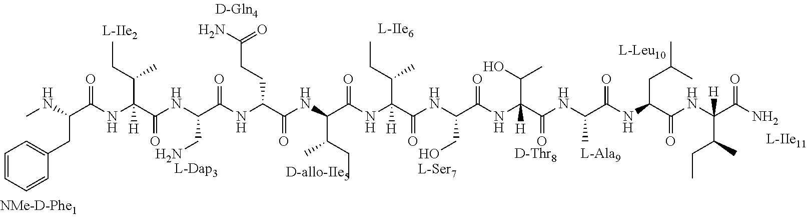 [SMILES: CC[C@H](C)[C@H](NC(=O)[C@H](CC(C)C)NC(=O)[C@H](C)NC(=O)[C@H](NC(=O)[C@H](CO)NC(=O)[C@@H](NC(=O)[C@H](NC(=O)[C@@H](CCC(N)=O)NC(=O)[C@H](CN)NC(=O)[C@@H](NC(=O)[C@H](Cc1ccccc1)NC)[C@@H](C)CC)[C@@H](C)CC)[C@@H](C)CC)C(C)O)C(N)=O] | 21 |
| APL-063 | |
| 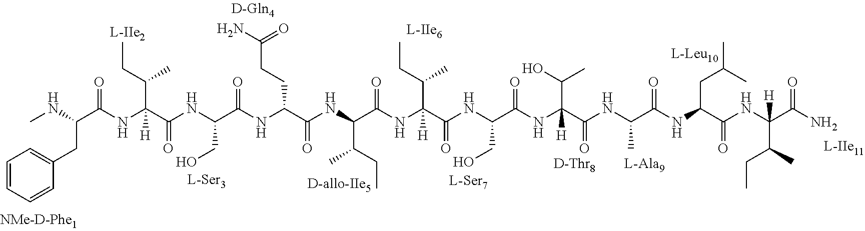 [SMILES: CC[C@H](C)[C@H](NC(=O)[C@H](CC(C)C)NC(=O)[C@H](C)NC(=O)[C@H](NC(=O)[C@H](CO)NC(=O)[C@@H](NC(=O)[C@H](NC(=O)[C@@H](CCC(N)=O)NC(=O)[C@H](CO)NC(=O)[C@@H](NC(=O)[C@H](Cc1ccccc1)NC)[C@@H](C)CC)[C@@H](C)CC)[C@@H](C)CC)C(C)O)C(N)=O] | 22 |
| APL-064 | |
| 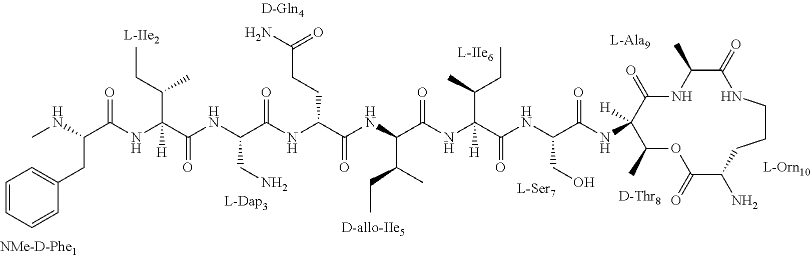 [SMILES: CC[C@H](C)[C@H](NC(=O)[C@H](Cc1ccccc1)NC)C(=O)N[C@@H](CN)C(=O)N[C@H](CCC(N)=O)C(=O)N[C@@H](C(=O)N[C@H](C(=O)N[C@@H](CO)C(=O)N[C@H]1C(=O)N[C@@H](C)C(=O)NCCC[C@H](N)C(=O)O[C@H]1C)[C@@H](C)CC)[C@@H](C)CC] | 23 |
| APL-065 | |
| 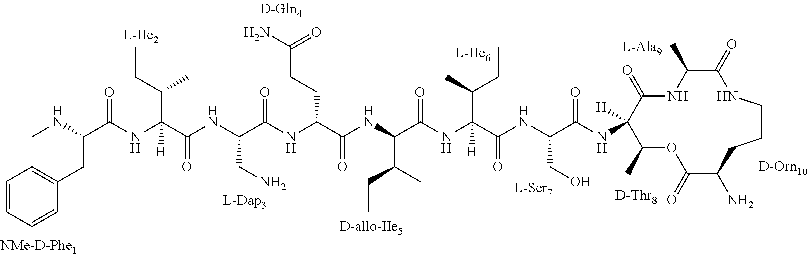 [SMILES: CC[C@H](C)[C@H](NC(=O)[C@H](Cc1ccccc1)NC)C(=O)N[C@@H](CN)C(=O)N[C@H](CCC(N)=O)C(=O)N[C@@H](C(=O)N[C@H](C(=O)N[C@@H](CO)C(=O)N[C@H]1C(=O)N[C@@H](C)C(=O)NCCC[C@@H](N)C(=O)O[C@H]1C)[C@@H](C)CC)[C@@H](C)CC] | 24 |
| APL-066 | |

The foregoing examples are illustrative of the present invention and are not to be construed as limiting thereof. Although the invention has been described in detail with reference to preferred embodiments, variations and modifications exist within the scope and spirit of the invention as described and defined in the following claims.

1. A teixobactin-antibiotic hybrid molecule comprising teixobactin covalently linked to an antibiotic.

2. The molecule of claim 1, wherein the antibiotic is covalently linked to the antibiotic by a linker.

3. The molecule of claim 1, wherein the antibiotic is a lipopeptide antibiotic, dalavancin, vancomycin, orivancin, telavancin or a carbapenem.

4. The molecule of claim 3, wherein the lipopeptide antibiotic is a cyclic lipopeptide antibiotic.

5. The molecule of claim 3, wherein the lipopeptide antibiotic is a polymyxin or derivative thereof, daptomycin, or an octapeptin.

6. The molecule of claim 5, wherein the polymyxin is polymyxin B.

7. The molecule of claim 5, wherein the polymyxin derivative is polymyxin B nonapeptide, FADDI-002 or FADD-115.

8. The molecule of claim 5, wherein the octapeptin is octapeptin C4

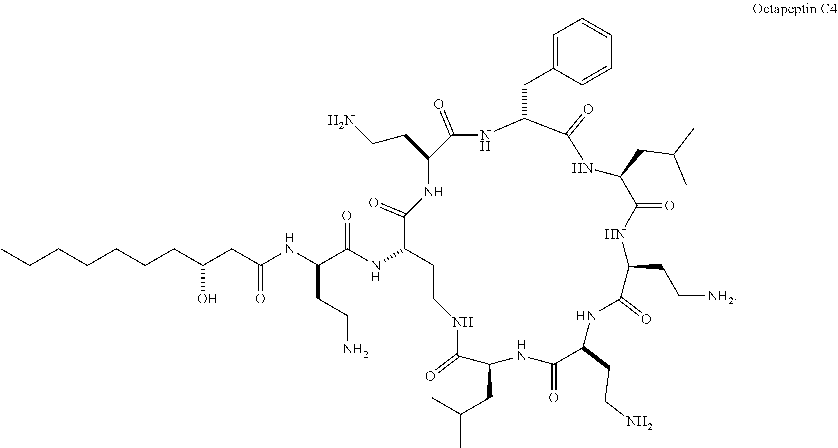

9. The molecule of claim 2, wherein the linker is a non-labile linker.

10. The molecule of claim 2, wherein the linker is a labile linker.

11. The molecule of claim 10, wherein the labile linker comprises a disulfide bond.

12. The molecule of claim 1, having the structure:

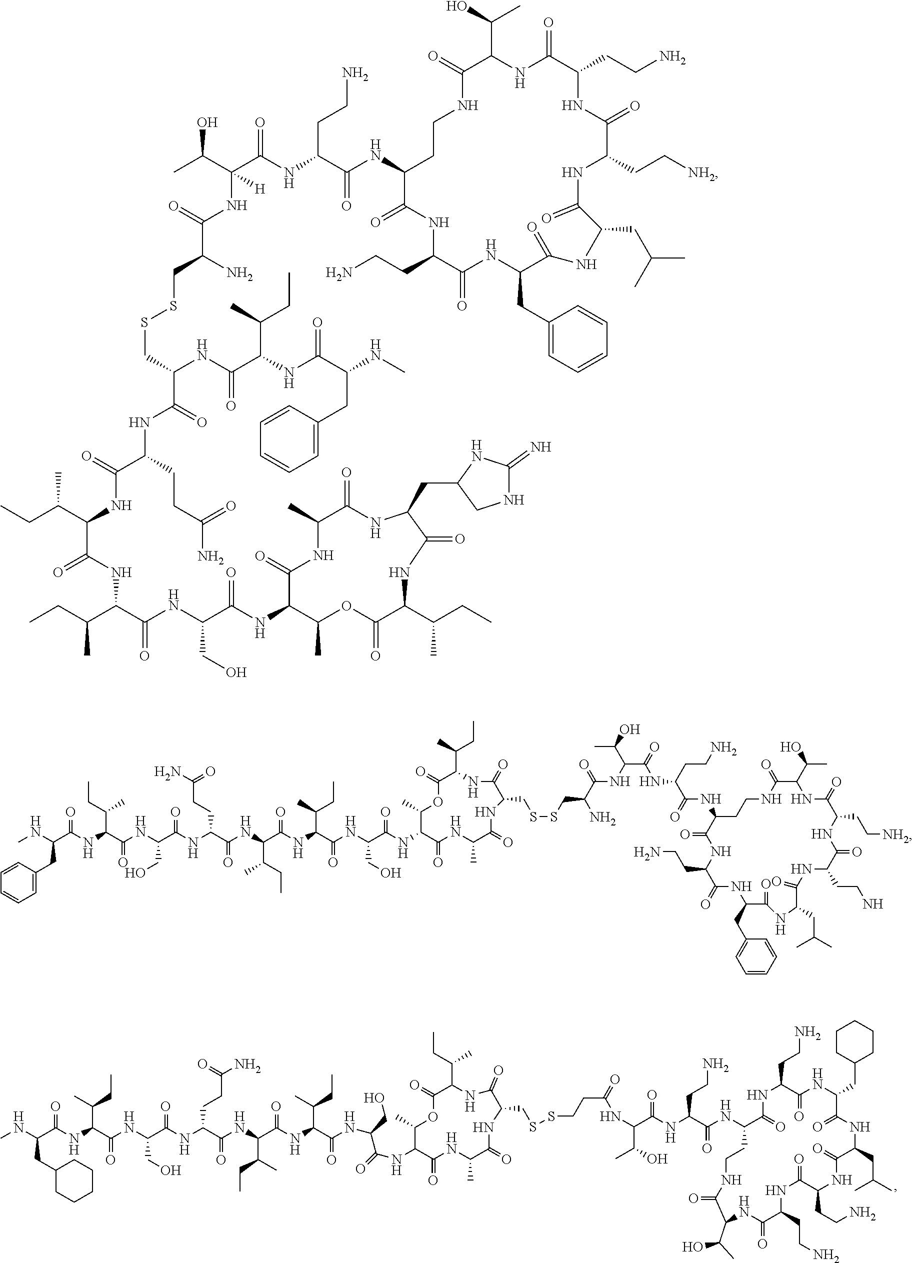

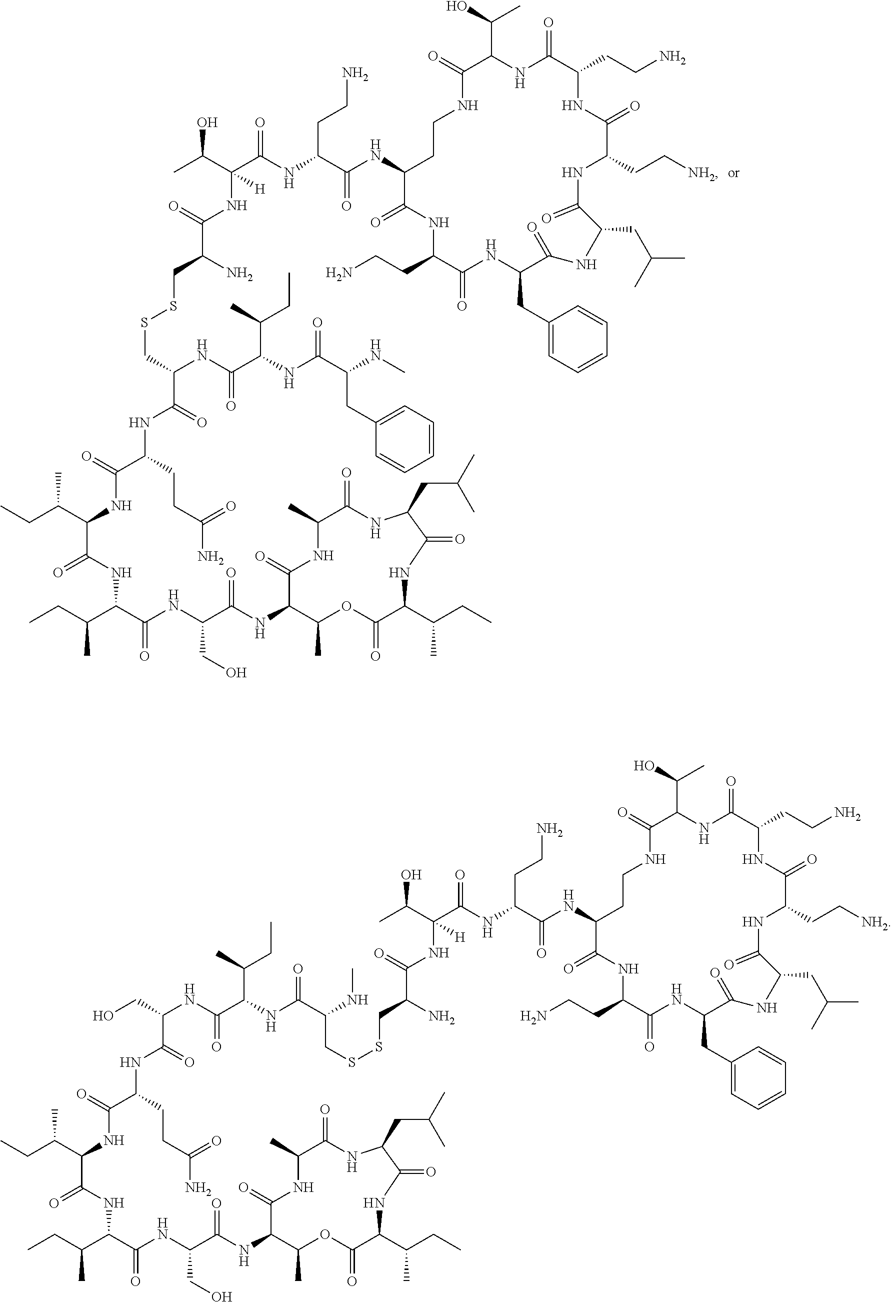

13. The molecule of claim 1, wherein the teixobactin comprises one or more modifications from Table 5.

14. A pharmaceutical composition comprising the molecule of claim 1 and optionally comprising one or more excipients, optionally wherein the pharmaceutical composition is formulated for inhalation, as a dry powder for inhalation, as a hydrogel, or for topical administration.

15-18. (canceled)

19. A wound dressing comprising the pharmaceutical composition of claim 14.

20. A method of treating a bacterial infection in a subject in need thereof, comprising administering to the subject a therapeutically effective amount of the molecule of claim 1, thereby treating the bacterial infection in the subject.

21-27. (canceled)

28. A method of treating a bacterial lung infection in a subject in need thereof, comprising administering to the subject a therapeutically effective amount of the molecule of claim 1, thereby treating the bacterial lung infection in the subject.

29-31. (canceled)

32. A method of treating a bacterial infection in a subject in need thereof, comprising administering to the subject a therapeutically effective amount of a topical composition comprising the molecule of claim 1, thereby treating the bacterial infection in the subject.

33-37. (canceled)

37. The method of claim 20, wherein the administration is prior to identification of the bacterial infection as Gram positive or Gram negative.

38. The method of claim 20, wherein the bacteria do not develop resistance to the molecule or composition.


SEQUENCE LISTING (24 sequences)

SEQ ID NO: 1 - protein, 11 aa, synthetic construct
  SITE 1: note = N-Methyl-D-Phenylalanine
  SITE 4: note = D-Glutamine
  SITE 5: note = Allo-Isoleucine
  SITE 8: note = D-Threonine
  SITE 10: note = Allo-Enduracididine
  REGION 8..11: note = Cyclopeptide
FISQIISTAXI

SEQ ID NO: 2 - protein, 9 aa, synthetic construct
  SITE 2..4: note = L-Diaminobutyric acid
  SITE 5: note = D-Phenylalanine
  SITE 7..8: note = L-Diaminobutyric acid
  REGION 3..9: note = Cyclopeptide
TXXXFLXXT

SEQ ID NO: 3 - protein, 10 aa, synthetic construct
  SITE 1: note = N-Boc-N-Methyl-D-Phenylalanine
  SITE 4: note = D-Glutamine
  SITE 5: note = D-Isoleucine
  SITE 8: note = D-Threonine
FISQIISTAL

SEQ ID NO: 4 - protein, 10 aa, synthetic construct
  SITE 1: note = N-Boc-N-Methyl-D-Phenylalanine
  SITE 4: note = D-Glutamine
  SITE 5: note = D-Isoleucine
  SITE 8: note = D-Threonine
  SITE 8: note = Side chain linkage to Isoleucine
FISQIISTAL

SEQ ID NO: 5 - protein, 11 aa, synthetic construct
  SITE 1: note = N-Methyl-D-Phenylalanine
  SITE 4: note = D-Glutamine
  SITE 5: note = D-Isoleucine
  SITE 8: note = D-Threonine
  REGION 8..11: note = Cyclopeptide
FISQIISTALI

SEQ ID NO: 6 - protein, 11 aa, synthetic construct
  SITE 1: note = N-Methyl-D-Phenylalanine
  SITE 5: note = D-Allo-Isoleucine
  SITE 8: note = D-Threonine
  REGION 8..11: note = Cyclopeptide
FIXXIIXTALI

SEQ ID NO: 7 - protein, 11 aa, synthetic construct
  SITE 1: note = N-Methyl-D-Phenylalanine
  SITE 4: note = D-Glutamine
  SITE 5: note = D-Allo-Isoleucine
  SITE 8: note = D-Threonine
  SITE 10: note = Linked to Polymyxin B via disulfide bond
  REGION 8..11: note = Cyclopeptide
FISQIISTACI

SEQ ID NO: 8 - protein, 9 aa, synthetic construct
  SITE 1: note = Linked to Teixobactin via disulfide bond
  SITE 2..4: note = L-Diaminobutyric acid
  SITE 5: note = D-Phenylalanine
  SITE 7..8: note = L-Diaminobutyric acid
  REGION 3..9: note = Cyclopeptide
TXXXFLXXT

SEQ ID NO: 9 - protein, 10 aa, synthetic construct
  SITE 1: note = 6-Methylheptanoyl-L-Diaminobutyric acid
  SITE 3..5: note = L-Diaminobutyric acid
  SITE 6: note = D-Leucine
  SITE 8..9: note = L-Diaminobutyric acid
  REGION 4..10: note = Cyclopeptide
XTXXXLLXXT

SEQ ID NO: 10 - protein, 10 aa, synthetic construct
  SITE 1: note = 6-Methyloctanoyl-L-Diaminobutyric acid
  SITE 3..5: note = L-Diaminobutyric acid
  SITE 6: note = D-Leucine
  SITE 8..9: note = L-Diaminobutyric acid
  REGION 4..10: note = Cyclopeptide
XTXXXLLXXT

SEQ ID NO: 11 - protein, 11 aa, synthetic construct
  SITE 1: note = N-Methyl-D-Phenylalanine
  SITE 4: note = D-Diaminobutyric acid
  SITE 5: note = D-Allo-Isoleucine
  SITE 8: note = D-Threonine
  REGION 8..11: note = Cyclopeptide
FISXIISTALI

SEQ ID NO: 12 - protein, 11 aa, synthetic construct
  SITE 1: note = N-Methyl-D-Phenylalanine
  SITE 3: note = L-Diaminopropionic acid
  SITE 4: note = D-Glutamine
  SITE 5: note = D-Allo-Isoleucine
  SITE 8: note = D-Threonine
  REGION 8..11: note = Cyclopeptide
FIXQIISTALI

SEQ ID NO: 13 - protein, 11 aa, synthetic construct
  SITE 1: note = N-Methyl-D-Phenylalanine
  SITE 4: note = D-Lysine
  SITE 5: note = D-Allo-Isoleucine
  SITE 8: note = D-Threonine
  REGION 8..11: note = Cyclopeptide
FISKIISTALI

SEQ ID NO: 14 - protein, 11 aa, synthetic construct
  SITE 1: note = N-Methyl-D-Phenylalanine
  SITE 4: note = D-Glutamine
  SITE 5: note = D-Allo-Isoleucine
  SITE 7: note = L-Diaminopropionic acid
  SITE 8: note = D-Threonine
  REGION 8..11: note = Cyclopeptide
FISQIIXTALI

SEQ ID NO: 15 - protein, 11 aa, synthetic construct
  SITE 1: note = N-Methyl-D-Phenylalanine
  SITE 4: note = D-Glutamine
  SITE 5: note = D-Allo-Isoleucine
  SITE 8: note = D-Diaminopropionic acid
  REGION 8..11: note = Cyclopeptide
FISQIISXALI

SEQ ID NO: 16 - protein, 11 aa, synthetic construct
  SITE 1: note = N-Methyl-D-Phenylalanine
  SITE 3: note = L-Diaminopropionic acid
  SITE 4: note = D-Glutamine
  SITE 5: note = D-Allo-Isoleucine
  SITE 8: note = D-Diaminopropionic acid
  REGION 8..11: note = Cyclopeptide
FIXQIISXALI

SEQ ID NO: 17 - protein, 11 aa, synthetic construct
  SITE 1: note = N-Methyl-D-Phenylalanine
  SITE 4: note = D-Glutamine
  SITE 5: note = D-Allo-Isoleucine
  SITE 8: note = D-Diaminopropionic acid
  REGION 8..11: note = Cyclopeptide
FISQIISXALL

SEQ ID NO: 18 - protein, 11 aa, synthetic construct
  SITE 1: note = N-Methyl-D-Phenylalanine
  SITE 3: note = L-Diaminopropionic acid
  SITE 4: note = D-Glutamine
  SITE 5: note = D-Allo-Isoleucine
  SITE 8: note = D-Diaminopropionic acid
  REGION 8..11: note = Cyclopeptide
FIXQIISXALL

SEQ ID NO: 19 - protein, 11 aa, synthetic construct
  SITE 1: note = N-Methyl-D-Phenylalanine
  SITE 4: note = D-Glutamine
  SITE 5: note = D-Allo-Isoleucine
  SITE 8: note = D-Diaminopropionic acid
FISQIISXALI

SEQ ID NO: 20 - protein, 11 aa, synthetic construct
  SITE 1: note = N-Methyl-D-Phenylalanine
  SITE 3: note = L-Diaminopropionic acid
  SITE 4: note = D-Glutamine
  SITE 5: note = D-Allo-Isoleucine
  SITE 8: note = D-Diaminopropionic acid
FIXQIISXALI

SEQ ID NO: 21 - protein, 11 aa, synthetic construct
  SITE 1: note = N-Methyl-D-Phenylalanine
  SITE 3: note = L-Diaminopropionic acid
  SITE 4: note = D-Glutamine
  SITE 5: note = D-Allo-Isoleucine
  SITE 8: note = D-Threonine
FIXQIISTALI

SEQ ID NO: 22 - protein, 11 aa, synthetic construct
  SITE 1: note = N-Methyl-D-Phenylalanine
  SITE 4: note = D-Glutamine
  SITE 5: note = D-Allo-Isoleucine
  SITE 8: note = D-Threonine
FISQIISTALI

SEQ ID NO: 23 - protein, 10 aa, synthetic construct
  SITE 1: note = N-Methyl-D-Phenylalanine
  SITE 3: note = L-Diaminopropionic acid
  SITE 4: note = D-Glutamine
  SITE 5: note = D-Allo-Isoleucine
  SITE 8: note = D-Threonine
  SITE 10: note = L-Ornithine
  REGION 8..10: note = Cyclopeptide
FIXQIISTAX

SEQ ID NO: 24 - protein, 10 aa, synthetic construct
  SITE 1: note = N-Methyl-D-Phenylalanine
  SITE 3: note = L-Diaminopropionic acid
  SITE 4: note = D-Glutamine
  SITE 5: note = D-Allo-Isoleucine
  SITE 8: note = D-Threonine
  SITE 10: note = D-Ornithine
  REGION 8..10: note = Cyclopeptide
FIXQIISTAX